(19) United States
(12) Patent Application Publication
(10) Pub. No.: US 2008/0188448 A1
(43) Pub. Date: Aug. 7, 2008

(54) NON-STEROIDAL PROGESTERONE RECEPTOR MODULATORS

(76) Inventors: Wolfgang Schwede, Glienicke (DE); Thomas Andrew Kirkland, Canyon, CA (US); Anja Schmidt, Berlin (DE); Ulrike Fuhrmann, Berlin (DE); Carsten Moeller, Berlin (DE); Andrea Rotgeri, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/961,360

(22) Filed: Dec. 20, 2007

Related U.S. Application Data
(60) Provisional application No. 60/880,706, filed on Jan. 17, 2007.
(60) Provisional application No. 60/944,870, filed on Jun. 19, 2007.
(60) Provisional application No. 60/938,338, filed on May 16, 2007.

(30) Foreign Application Priority Data
Dec. 21, 2006 (DE) 10 2006 061 913.7

(51) Int. Cl. A61K 31/536; C07D 265/02; A61P 35/00; A61P 15/00; A61K 31/56

(52) U.S. Cl. 514/171; 514/228.8; 544/66

(57) ABSTRACT

The present invention relates to non-steroidal progesterone receptor modulators of the general formula I,

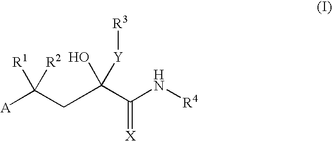

the use of the progesterone receptor modulators for the manufacture of medicaments, and pharmaceutical compositions which comprise these compounds.

The compounds according to the invention are suitable for the therapy and prophylaxis of gynaecological disorders such as endometriosis, leiomyomas of the uterus, dysfunctional bleeding and dysmenorrhoea, and for the therapy and prophylaxis of hormone-dependent tumours and for use for female fertility control and for hormone replacement therapy.

This application claims the benefit of the filing date of U.S. Provisional Application Ser. No. 60/880,706 filed Jan. 17, 2007, of U.S. Provisional Application Ser. No. 60/944,870 filed Jun. 19, 2007, and of U.S. Provisional Application Ser. No. 60/938,338 filed May 16, 2007.

The present invention relates to non-steroidal progesterone receptor modulators, a method for their preparation, the use of the progesterone receptor modulators for the manufacture of medicaments, and pharmaceutical compositions which comprise these compounds.

The steroid hormone progesterone controls in a decisive manner the reproductive process in the female body. Progesterone is secreted in large quantities during the cycle and pregnancy respectively by the ovary and the placenta. Progesterone in cooperation with oestrogens brings about cyclic changes in the uterine mucosa (endometrium) during the menstrual cycle. Elevated progesterone levels after ovulation influence the uterine mucosa to convert it into a state permitting nidation of an embryo (blastocyst). During pregnancy, progesterone controls the relaxation of the myometrium and maintains the function of the decidual tissue.

It is further known that progesterone inhibits endometrial proliferation by suppressing oestrogen-mediated mitosis in uterine tissue (K. Chwalisz, R. M. Brenner, U. Fuhrmann, H. Hess-Stumpp, W. Elger, Steroids 65, 2000, 741-751).

Progesterone and progesterone receptors are also known to play a significant part in pathophysiological processes. Progesterone receptors have been detected in the foci of endometriosis, but also in tumours of the uterus, of the breast and of the CNS. It is further known that uterine leiomyomas grow progesterone-dependently.

The effects of progesterone in the tissues of the genital organs and in other tissues occur through interactions with progesterone receptors which are responsible for the cellular effects.

Progesterone receptor modulators are either pure agonists or inhibit the effect of progesterone partly or completely. Accordingly, substances are defined as pure agonists, partial agonists (selective progesterone receptor modulators=SPRMS) and pure antagonists.

In accordance with the ability of progesterone receptor modulators to display their effect via the progesterone receptor, these compounds have a considerable potential as therapeutic agents for gynaecological and oncological indications and for obstetrics and fertility control.

Pure progesterone receptor antagonists completely inhibit the effect of progesterone on the progesterone receptor. They have anti-ovulatory properties and the ability to inhibit oestrogen effects in the endometrium, as far as complete atrophy. They are therefore particularly suitable for intervening in the female reproductive process, e.g. post-ovulation, in order to prevent nidation of a fertilized egg cell, during pregnancy in order to increase the reactivity of the uterus to prostaglandins or oxytocin, or in order to achieve opening and softening (“ripening”) of the cervix, and to induce a great readiness of the myometrium to contract.

A beneficial effect on the pathological event is expected in foci of endometriosis and in tumour tissues which are equipped with progesterone receptors after administration of pure progesterone receptor antagonists. There might be particular advantages for influencing pathological states such as endometriosis or uterine leiomyomas if ovulation inhibition can additionally be achieved by the progesterone receptor antagonists. Ovulation inhibition also dispenses with some of the ovarian hormone production and thus the stimulating effect, deriving from this proportion, on the pathologically altered tissue.

The first progesterone receptor antagonist described, RU 486 (also mifepristone), was followed by the synthesis and characterization of a large number of analogues with progesterone receptor-antagonistic activity of varying strength. Whereas RU 486 also shows an antiglucocorticoid effect in addition to the progesterone receptor-antagonistic effect, compounds synthesized later are notable in particular for a more selective effect as progesterone receptor antagonists.

Besides steroidal compounds such as onapristone or lilopristone, which are notable by comparison with RU 486 for a better dissociation of the progesterone receptor-antagonistic effect and the antiglucocorticoid effect, also known from the literature are various non-steroidal structures whose antagonistic effect on the progesterone receptor is being investigated [see, for example, S. A. Leonhardt and D. P. Edwards, Exp. Biol. Med. 227: 969-980 (2002) and R. Winneker, A. Fensome, J. E. Wrobel, Z. Zhang, P. Zhang, Seminars in Reproductive Medicine, Volume 23: 46-57 (2005)]. However, non-steroidal compounds disclosed to date have only moderate antagonistic activity compared with the activity of known steroidal structures. The most effective non-steroidal compounds are reported to have in vitro activities which are 10% of the activity of RU 486.

The antiglucocorticoid activity is disadvantageous for therapeutic use, where the inhibition of progesterone receptors is at the forefront of the therapy. An antiglucocorticoid activity causes unwanted side effects at the dosages necessary for therapy. This may prevent administration of a therapeutically worthwhile dose or lead to discontinuation of the treatment.

Partial or complete reduction of the antiglucocorticoid properties is therefore an important precondition for therapy with progesterone receptor antagonists, especially for those indications requiring treatment lasting weeks or months.

In contrast to the pure antagonists, partial progesterone receptor agonists (SPRMs) show a residual agonistic property which may vary in strength. This leads to these substances showing agonistic effects on the progesterone receptor in certain organ systems (D. DeManno, W. Elger, R. Garg, R. Lee, B. Schneider, H. Hess-Stumpp, G. Schuber, K. Chwalisz, Steroids 68, 2003, 1019-1032). Such an organ-specific and dissociated effect may be of therapeutic benefit for the described indications.

It is therefore an object of the present invention to provide further non-steroidal progesterone receptor modulators. These compounds are intended to have a reduced antiglucocorticoid effect and therefore be suitable for the therapy and prophylaxis of gynaecological disorders such as endometriosis, leiomyomas of the uterus, dysfunctional bleeding and dysmenorrhoea. The compounds according to the invention are additionally intended to be suitable for the therapy and prophylaxis of hormone-dependent tumours, for example of breast, endometrial, ovarian and prostate carcinomas. The compounds are intended furthermore to be suitable for use in female fertility control and for female hormone replacement therapy.

The object is achieved according to the present invention by the provision of non-steroidal compounds of the general formula I

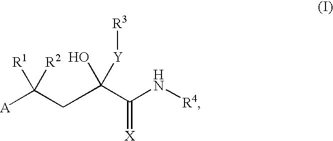

in which
A is hydrogen, a C₁-C₈-alkyl, C₂-C₈-alkenyl, C₂-C₈-alkynyl radical which is optionally mono- or polysubstituted identically or differently by Z, or a C₃-C₁₀-cycloalkyl or 3-12-membered heterocycloalkyl radical which is optionally mono- or polysubstituted identically or differently by M, or else is Z itself, where Z is defined as follows:
   cyano, halogen, hydroxyl, nitro, —C(O)R^{b}, CO₂R^{b}, —O—R^{b}, —S—R^{b}, SO₂NR^{c}R^{d}, —C(O)—NR^{c}R^{d}, —OC(O)—NR^{c}R^{d}, —C═NOR^{b}, —NR^{c}R^{d}, —PO₃(R^{b})₂, —NR^{e}COR^{b}, —NR^{e}CSR^{b}, —NR^{e}S(O)R^{b}, —NR^{e}S(O)₂R^{b}, —NR^{e}CONR^{c}R^{d}, —NR^{e}COOR^{b}, —NR^{e}C(NH)NR^{c}R^{d}, —NR^{e}CSNR^{c}R^{d}, —NR^{e}S(O)NR^{c}R^{d}, —NR^{e}S(O)₂NR^{c}R^{d}, —S(O)R^{b}, —S(O)NR^{c}R^{d}, —S(O)₂R^{b}, —SO₂OR^{b}, —CSNR^{c}R^{d}, —CR^{b}(OH)—R^{b} or
   a C₃-C₁₀-cycloalkyl or 3-12-membered heterocycloalkyl radical which is optionally mono- or polysubstituted identically or differently by M and
      M is C₁-C₆-alkyl or a —COR^{b}, CO₂R^{b}, —O—R^{b} or —NR^{c}R^{d} group, where
      R^{b} is hydrogen or a C₁-C₆-alkyl, C₂-C₈-alkenyl, C₂-C₈-alkynyl, C₃-C₁₀-cycloalkyl, C₆-C₁₂-aryl or a partly or fully fluorinated C₁-C₃-alkyl radical and
      R^{c} and R^{d} are each independently hydrogen, a C₁-C₆-alkyl, C₂-C₈-alkenyl, C₂-C₈-alkynyl, C₃-C₁₀-cycloalkyl or C₆-C₁₂-aryl radical, a C(O)R^{b} group where R^{b} is as defined above or a hydroxyl group,
      where, when
      R^{c} is a hydroxyl group, R^{d} can only be hydrogen, C₁-C₆-alkyl, C₂-C₈-alkenyl, C₂-C₈-alkynyl, C₃-C₁₀-cycloalkyl or C₆-C₁₂-aryl, and vice versa, and also
      R^{e} is hydrogen, C₁-C₆-alkyl, C₂-C₈-alkenyl, C₂-C₈-alkynyl, C₃-C₁₀-cycloalkyl or C₆-C₁₂-aryl, and
R¹ and R² are each independently an unbranched or branched C₁-C₅-alkyl group or, together with the carbon atom of the chain, form a ring having a total of 3-7 members,
   where, when
   A is hydrogen, R¹ and R² cannot both be a methyl radical,
R³ is hydrogen or a C₁-C₈-alkyl, C₂-C₈-alkenyl, C₂-C₈-alkynyl, C₃-C₁₀-cycloalkyl, 3-12-membered heterocycloalkyl radical which is optionally mono- or polysubstituted identically or differently by K, or a mono- or bicyclic C₆-C₁₂-aryl or 3-12-membered heteroaryl radical which is optionally mono- or polysubstituted identically or differently by L, and
   K is cyano, halogen, hydroxyl, nitro, —C(O)R^{b}, CO₂R^{b}, —O—R^{b}, —S—R^{b}, SO₂NR^{c}R^{d}, —C(O)—NR^{c}R^{d}, —OC(O)—NR^{c}R^{d}, —C═NOR^{b}, —NR^{c}R^{d} or a C₃-C₁₀-cycloalkyl, 3-12-membered heterocycloalkyl radical which is optionally mono- or polysubstituted identically or differently by M, or a C₆-C₁₂-aryl or 3-12-membered heteroaryl radical which is optionally mono- or polysubstituted by L, with the definition of M specified under A, and
   L is C₁-C₈-alkyl, C₂-C₈-alkenyl, C₂-C₈-alkynyl, a partly or fully fluorinated C₁-C₆-alkyl, a partly or fully fluorinated C₁-C₆-alkoxy, C₁-C₆-alkoxy-C₁-C₆-alkyl, C₁-C₆-alkoxy-C₁-C₆-alkoxy, a mono- or bicyclic (CH₂)ₚ—C₃-C₁₀-cycloalkyl, a mono- or bicyclic 3-12-membered (CH₂)ₚ-heterocycloalkyl radical, (CH₂)ₚCN, (CH₂)ₚHal, (CH₂)ₚNO₂, a mono- or bicyclic (CH₂)ₚ—C₆-C₁₂-aryl radical, a mono- or bicyclic 3-12-membered (CH₂)ₚ-heteroaryl radical, or
      —(CH₂)pPO3(Rb)2, —(CH2)pNRcRd, —(CH2)pNReCORb, —(CH2)pNReCSRb, —(CH2)pNReS(O)Rb, —(CH2)pNReS(O)2Rb, —(CH2)pNReCONRcRd, —(CH2)pNReCOORb, —(CH2)pNReC(NH)NRcRd, —(CH2)pNReCSNRcRd, —(CH2)pNReS(O)NRcRd, —(CH2)pNReS(O)2NRcRd, —(CH2)pCORb, —(CH2)pCSRb, —(CH2)p S(O)Rb, —(CH2)pS(O)(NH)Rb, —(CH2)pS(O)2Rb, —(CH2)pS(O)2NRcRd, —(CH2)pSO2ORb, —(CH2)pCO2Rb, —(CH2)pCONRcRd, —(CH2)pCSNRcRd, —(CH2)pORb, —(CH2)pSRb, —(CH2)pCRb(OH)—Rb, —(CH₂)ₚ—C═NOR^{b}, —O—(CH₂)ₙ—O—, —O—(CH₂)ₙ—CH₂—, —O—CH═CH— or —(CH₂)ₙ₊₂—, and the terminal oxygen atoms and/or carbon atoms are linked to directly adjacent ring carbon atoms, and
   n is 1 or 2 and
   p is0, 1, 2, 3, 4, 5 or 6, and
X is one oxygen atom or two hydrogen atoms
Y is (CH₂)ₘ, —C≡C— or —CH═CH— where m=0 or 1, and
R⁴ is an aromatic or heteroaromatic 3- to 12-membered mono- or bicycle which is unsubstituted or optionally substituted by from 1 to 3 of the radicals mentioned under L, or one of the following groups mentioned under B or C:

### B: 6-Membered/6-Membered Ring Systems

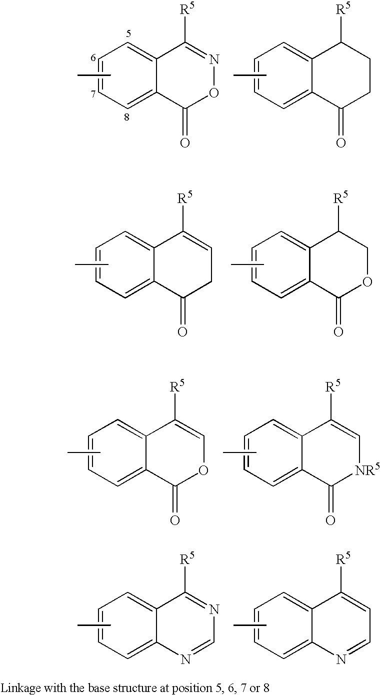

### C: 6-Membered/5-Membered Ring Systems

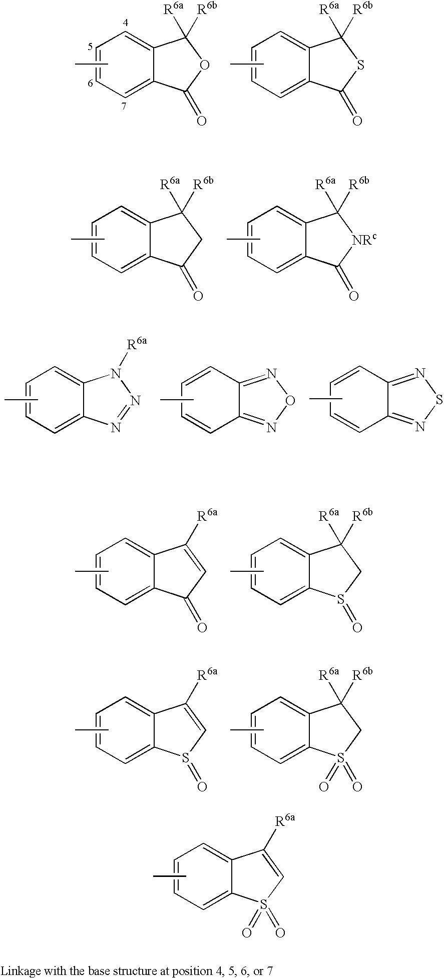 [0042] where
R⁵ is hydrogen or C₁-C₄-alkyl, or a partly or fully fluorinated C₁-C₄-alkyl,
R^{6a} and R^{6b} are each independently hydrogen, C₁-C₄-alkyl or a partly or fully fluorinated C₁-C₄-alkyl, or, together with the ring carbon atom, form a 3- to 6-membered ring,

and the pharmaceutically acceptable salts thereof.

The compounds according to the invention of the general formula I may, owing to the presence of centres of asymmetry, exist as different stereoisomers. Both the racemates and the separate stereoisomers belong to the subject matter of the present invention.

The present invention further includes the novel compounds as active pharmaceutical ingredients, their therapeutic use and pharmaceutical dosage forms which comprise the novel substances.

The compounds according to the invention of the general formula (I) or their pharmaceutically acceptable salts can be used to produce a medicament, in particular for the treatment and prophylaxis of gynaecological disorders such as endometriosis, leiomyomas of the uterus, dysfunctional bleeding and dysmenorrhoea. The compounds according to the invention may further be used for the treatment and prophylaxis of hormone-dependent tumours such as, for example, for breast, prostate and endometrial carcinoma.

The compounds according to the invention of the general formula (I) or their pharmaceutically acceptable salts are also suitable for use for female fertility control or for female hormone replacement therapy.

The non-steroidal compounds according to the invention of the general formula I have strong antagonistic or strong partial agonistic effects on the progesterone receptor with high potency. They show a strong dissociation of effects in relation to their strength of binding to the progesterone receptor and to the glucocorticoid receptor. Whereas known progesterone receptor antagonists such as mifepristone (RU 486) show, besides the desired high binding affinity for the progesterone receptor, likewise a high affinity for the glucocorticoid receptor, the compounds according to the invention are notable for a very low glucocorticoid receptor binding with simultaneously a high progesterone receptor affinity.

The substituents, defined as groups, of the compounds according to the invention of the general formula I may in each case have the following meanings:

C₁-C₄—, C₁-C₅—, C₁-C₆— and C₁-C₈-alkyl group means unbranched or optionally branched alkyl radicals. Examples thereof are a methyl, ethyl, n-propyl, isopropyl, n-, iso-, tert-butyl, an n-pentyl, 2,2-dimethylpropyl, 3-methylbutyl, hexyl, heptyl or octyl group. In the meaning of R¹, R² and R³, the methyl, ethyl, n-propyl or n-butyl group and an n-pentyl group are preferred.

According to the invention, preference is given to methyl or ethyl for R⁵, and to hydrogen for R^{6a} and R^{6b}.

Alkenyl means unbranched or optionally branched alkenyl radicals. Examples of the meaning of a C₂-C₈-alkenyl group in the context of the invention are the following: vinyl, allyl, 3-buten-1-yl or 2,3-dimethyl-2-propenyl. When the aromatic in R³ is substituted by a C₂-C₈-alkenyl radical, it is preferably a vinyl group.

Alkynyl means unbranched or optionally branched alkynyl radicals. A C₂-C₈-alkynyl radical is intended to be for example an ethynyl, propynyl, butynyl, pentynyl, hexynyl and octynyl group, but preferably an ethynyl or propynyl group.

Possible examples of C₁-C₆-alkoxyl-C₁-C₆-alkoxy group are methoxymethoxy, ethoxymethoxy or 2-methoxyethoxy.

A radical OR^{b} in the context of the invention is a hydroxy, methoxy, ethoxy, n-propoxy, isopropoxy, n-, iso-, tert-butoxy or n-pentoxy, 2,2-dimethylpropoxy or 3-methylbutoxy group. Hydroxy, methoxy and ethoxy are preferred.

Suitable for a partly or completely fluorinated C₁-C₄-alkyl group are in particular the trifluoromethyl or pentafluoroethyl group.

A halogen atom may be a fluorine, chlorine, bromine or iodine atom. Fluorine, chlorine or bromine is preferred here.

3- to 12-membered cycloalkyl and heterocycloalkyl groups are understood to mean both monocyclic and bicyclic groups.

Examples which may be mentioned of monocyclic C₃-C₁₀-cycloalkyl in the meaning of R^{c} and R^{e} are cyclopropane, cyclobutane, cyclopentane and cyclohexane. Cyclopropyl, cyclopentyl and cyclohexyl are preferred.

When R¹ and R² together with the carbon atom of the chain form a ring having a total of 3-7 members, this preferably means rings composed of a total of 3-7 carbon atoms. Particular preference is given to cyclopentyl and cyclohexyl when A is simultaneously hydrogen.

A mono- or bicyclic C₆-C₁₂-aryl radical in the meaning of R³ or R^{b}, R^{c}, R^{d}, R^{e}, and also K and L, is, for example, a phenyl or naphthyl radical, preferably a phenyl radical.

Examples of a 3-1 2-membered heteroaryl radical in the meaning of R³, K and L are the 2-, 3- or 4-pyridinyl, the 2- or 3-furyl, the 2- or 3-thienyl, the 2- or 3-pyrrolyl, the 2-, 4- or 5-imidazolyl, the pyrazinyl, the 2-, 4- or 5-pyrimidinyl, or the 3- or 4-pyridazinyl radical.

Examples of monocyclic 3-10-membered heterocyclic radicals in the meaning of A, Z, K, R³ or R⁴ are morpholine, tetrahydrofuran, piperidine, pyrrolidine oxirane, oxetane, aziridine, dioxolane, dioxane, thiophene, furan, pyran, pyrrole, imidazole, pyrazole, pyridine, pyrazine, pyrimidine, pyridazine, piperazine, thiazole, oxazole, furazane, pyrroline, thiazoline, triazole, tetrazole, using any of the chemically possible isomers in relation to the positions of the heteroatoms.

Examples which may be mentioned of bicyclic 3-10-membered heterocycles are quinoline, quinazoline and naphthyridine.

For R⁴, according to the invention, the bicyclic ring systems specified under B and C are preferred.

The number p for the (CH₂)ₚ radical may be a number 0, 1, 2, 3, 4, 5 or 6, preferably 0, 1 or 2. “Radical” means according to the invention all functional groups which are mentioned under L in connection with (CH₂)ₚ.

In the case where the compounds of the general formula I are in the form of salts, this is possible for example in the form of the hydrochloride, sulphate, nitrate, tartrate, citrate, fumarate, succinate or benzoate.

If the compounds according to the invention are in the form of racemic mixtures, they can be fractionated by methods of racemate resolution familiar to the skilled person into the pure optically active forms. For example, the racemic mixtures can be separated into the pure isomers by chromatography on a support material which is itself optically active (CHIRALPAK AD®). It is also possible to esterify the free hydroxy group in a racemic compound of the general formula I with an optically active acid, and to separate the resulting diastereoisomeric esters by fractional crystallization or chromatography and to hydrolyse the separated esters in each case to the optically pure isomers. It is possible to use as optically active acid for example mandelic acid, camphorsulphonic acid or tartaric acid.

Compounds of the general formula (I) preferred according to the present invention are those in which:
A is hydrogen and R¹ and R², together with the carbon atom of the chain, form a ring composed of 3-7 carbon atoms or
A is C₁-C₈-alkyl or C₃-C₁₀-cycloalkyl, while R¹ and R² are each methyl, or

and

with the prerequisite that Y is —C≡C— or —CH═CH—,
R³ is a C₁-C₈-alkyl, a mono- or bicyclic C₆-C₁₂-aryl or a 3-12-membered heteroaryl radical, or

when Y is (CH₂)ₘ,
R³ is a mono- or bicyclic C₆-C₁₂-aryl or a 3-12-membered heteroaryl and
R⁴ may be a mono- or disubstituted mono- or bicyclic aromatic or one of the B groups mentioned under R⁴ with linkage at position 6, or one of the C groups mentioned under R⁴ with linkage at position 5. Among these, the following are preferred in turn:

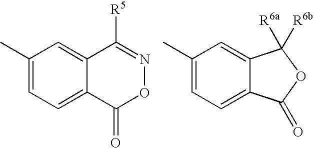

Preferably, in addition,
R⁵ is methyl or ethyl,
R⁶ is hydrogen,
p is 0, 1 or 2, and
L is C₁-C₈-alkyl, C₂-C₈-alkenyl, C₂-C₈-alkynyl, a partly or fully fluorinated C₁-C₆-alkyl, —(CH₂)ₚCN, (CH₂)ₚHal, (CH₂)ₚNO₂, (CH₂)ₚ—C₆-C₁₂-aryl, —(CH₂)ₚ-heteroaryl, —(CH₂)ₚNR^{c}R^{d}, —(CH₂)ₚNR^{e}COR^{b}, —(CH₂)ₚNR^{e}S(O)₂R^{b}, —(CH₂)ₚNR^{e}CONR^{c}R^{d}, (CH₂)ₚNR^{e}S(O)NR^{c}R^{d}, —(CH₂)ₚNR^{e}S(O)₂NR^{c}R^{d}, —(CH₂)ₚCOR^{b}, —(CH₂)ₚS(O)R^{b}, —(CH₂)ₚS(O)₂R^{b}, —(CH₂)ₚS(O)₂NR^{c}R^{d}, —(CH₂)ₚCO₂R^{b}, —(CH₂)ₚCONR^{c}R^{d}, —(CH₂)ₚOR^{b}, —(CH₂)ₚCR^{b}(OH)—R^{b} and
Z is cyano, halogen, hydroxyl, nitro, —C(O)R^{b}, CO₂R^{b}, —O—R^{b}, —SO₂NR^{c}R^{d}, —C(O)—NR^{c}R^{d}, —NR^{c}R^{d}, —NR^{e}COR^{b}, —NR^{e}S(O)R^{b}, —NR^{e}S(O)₂R^{b}, NR^{e}CONR^{c}R^{d}, —S(O)R^{b}, —S(O)NR^{c}R^{d}, —S(O)₂R^{b}, —CR^{b}(OH)—R^{b} or a C₃-C₁₀-cycloalkyl or heterocycloalkyl optionally mono- or polysubstituted identically or differently by M.

The compounds mentioned below, and the use thereof, are preferred according to the invention:

| | | |
|---|---|---|
| | Racemic or | |
| No. | enantiomer | —Y—R¹ |

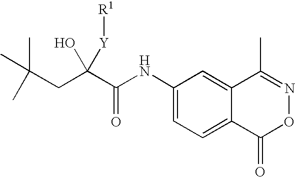

| | | |
|---|---|---|
| 123 | rac+− | 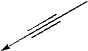 |
| 456 | rac+− | 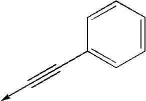 |
| 789 | rac+− | 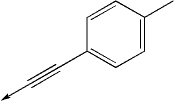 |
| 101112 | rac+− | 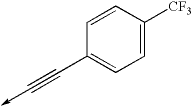 |
| 131415 | rac+− | 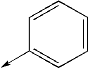 |
| 161718 | rac+− | 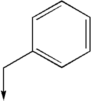 |
| 192021 | rac+− | 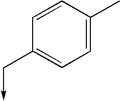 |
| 222324 | rac+− | 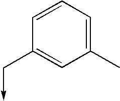 |

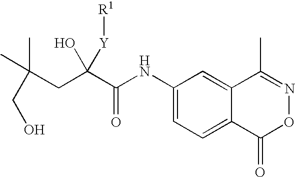

| | | |
|---|---|---|
| 252627 | rac+− | 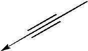 |
| 282930 | rac+− | 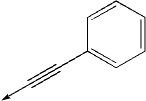 |
| 313233 | rac+− | 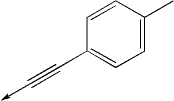 |
| 343536 | rac+− | 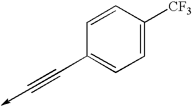 |
| 373839 | rac+− | 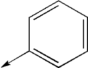 |
| 404142 | rac+− | 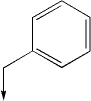 |
| 434445 | rac+− | 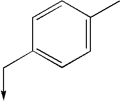 |
| 464748 | rac+− | 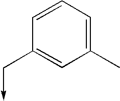 |

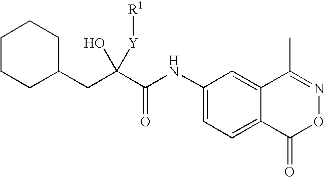

| | | |
|---|---|---|
| 495051 | rac+− | 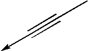 |
| 525354 | rac+− | 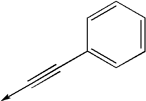 |
| 555657 | rac+− | 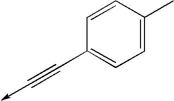 |
| 585960 | rac+− | 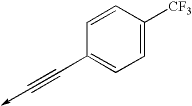 |
| 616263 | rac+− | 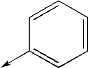 |
| 646566 | rac+− | 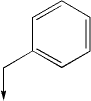 |
| 676869 | rac+− | 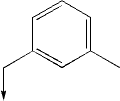 |
| 707172 | rac+− | 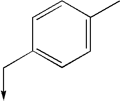 |

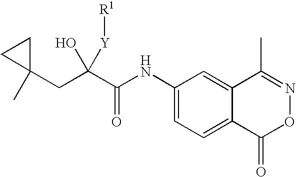

| | | |
|---|---|---|
| 737475 | rac+− | 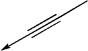 |
| 767778 | rac+− | 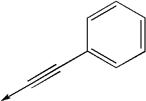 |
| 798081 | rac+− | 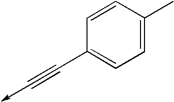 |
| 828384 | rac+− | 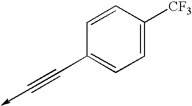 |
| 858687 | rac+− | 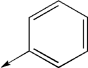 |
| 888990 | rac+− | 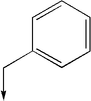 |
| 919293 | rac+− | 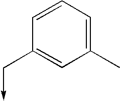 |
| 949596 | rac+− | 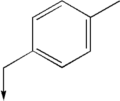 |

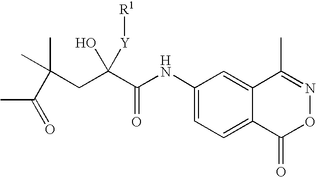

| | | |
|---|---|---|
| 979899 | rac+− | 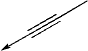 |
| 100101102 | rac+− | 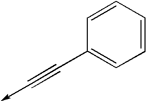 |
| 103104105 | rac+− | 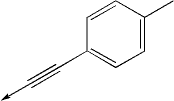 |
| 106107108 | rac+− | 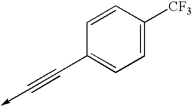 |
| 109110111 | rac+− | 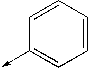 |
| 112113114 | rac+− | 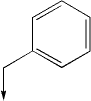 |
| 115116117 | rac+− | 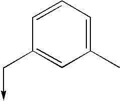 |
| 118119120 | rac+− | 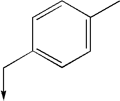 |

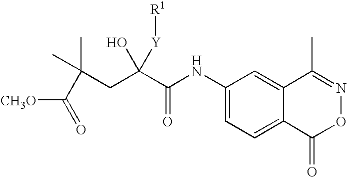

| | | |
|---|---|---|
| 121122123 | rac+− | 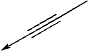 |
| 124125126 | rac+− | 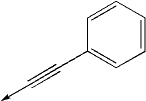 |
| 127128129 | rac+− | 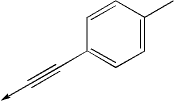 |
| 130131132 | rac+− | 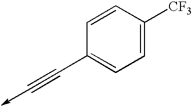 |
| 133134135 | rac+− | 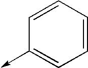 |
| 136137138 | rac+− | 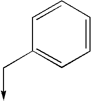 |
| 139140141 | rac+− | 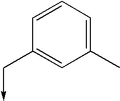 |
| 142143144 | rac+− | 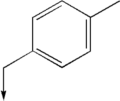 |

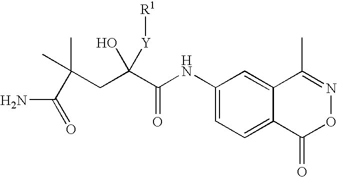

| | | |
|---|---|---|
| 145146147 | rac+− | 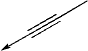 |
| 148149150 | rac+− | 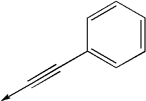 |
| 151152153 | rac+− | 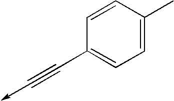 |
| 154155156 | rac+− | 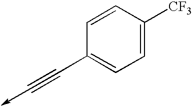 |
| 157158159 | rac+− | 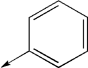 |
| 160161162 | rac+− | 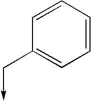 |
| 163164165 | rac+− | 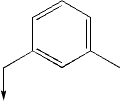 |
| 166167168 | rac+− | 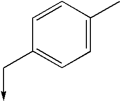 |

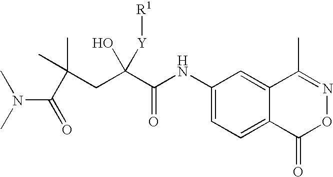

| | | |
|---|---|---|
| 169170171 | rac+− | 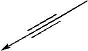 |
| 172173174 | rac+− | 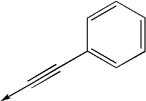 |
| 175176177 | rac+− | 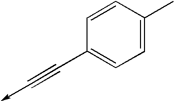 |
| 178179180 | rac+− | 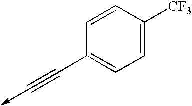 |
| 181182183 | rac+− | 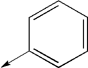 |
| 184185186 | rac+− | 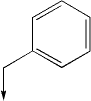 |
| 187188189 | rac+− | 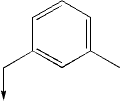 |
| 190191192 | rac+− | 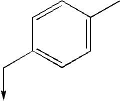 |

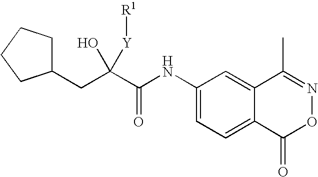

| | | |
|---|---|---|
| 193194195 | rac+− | 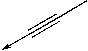 |
| 196197198 | rac+− | 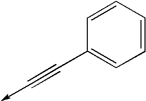 |
| 199200201 | rac+− | 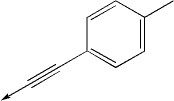 |
| 202203204 | rac+− | 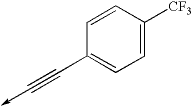 |
| 205206207 | rac+− | 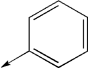 |
| 208209210 | rac+− | 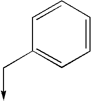 |
| 211212213 | rac+− | 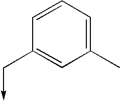 |
| 214215216 | rac+− | 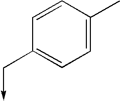 |

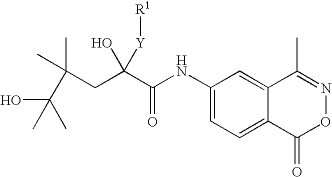

| | | |
|---|---|---|
| 217218219 | rac+− | 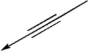 |
| 220221222 | rac+− | 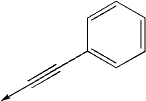 |
| 223224225 | rac+− | 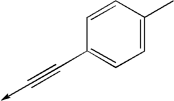 |
| 226227228 | rac+− | 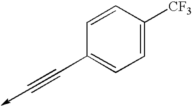 |
| 229230231 | rac+− | 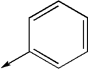 |
| 232233234 | rac+− | 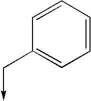 |
| 235236237 | rac+− | 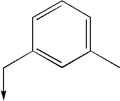 |
| 238239240 | rac+− | 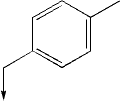 |

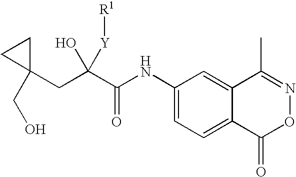

| | | |
|---|---|---|
| 241242243 | rac+− | 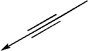 |
| 244245246 | rac+− | 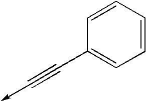 |
| 247248249 | rac+− | 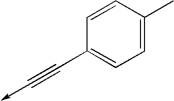 |
| 250251252 | rac+− | 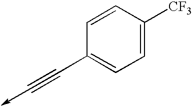 |
| 253254255 | rac+− | 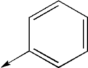 |
| 256257258 | rac+− | 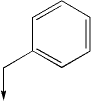 |
| 259260261 | rac+− | 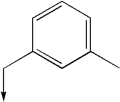 |
| 262263264 | rac+− | 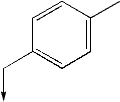 |

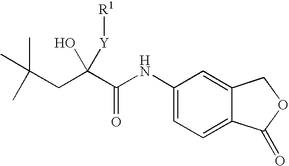

| | | |
|---|---|---|
| 265266267 | rac+− | 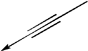 |
| 268269270 | rac+− | 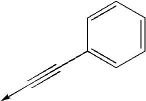 |
| 271272273 | rac+− | 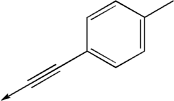 |
| 274275276 | rac+− | 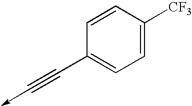 |
| 277278279 | rac+− | 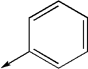 |
| 280281282 | rac+− | 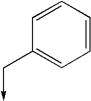 |
| 283284285 | rac+− | 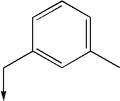 |
| 286287288 | rac+− | 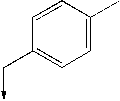 |

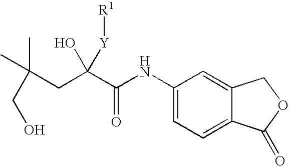

| | | |
|---|---|---|
| 289290291 | rac+− | 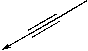 |
| 292293294 | rac+− | 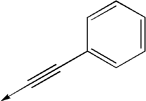 |
| 295296297 | rac+− | 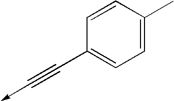 |
| 298299300 | rac+− | 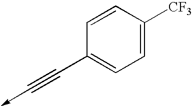 |
| 301302303 | rac+− | 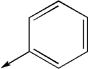 |
| 304305306 | rac+− | 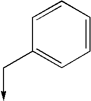 |
| 307308309 | rac+− | 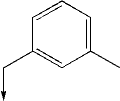 |
| 310311312 | rac+− | 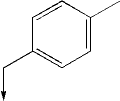 |

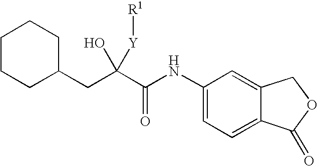

| | | |
|---|---|---|
| 313314315 | rac+− | 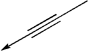 |
| 316317318 | rac+− | 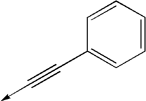 |
| 319320321 | rac+− | 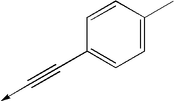 |
| 322323324 | rac+− | 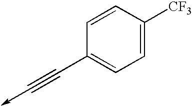 |
| 325326327 | rac+− | 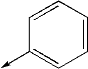 |
| 328329330 | rac+− | 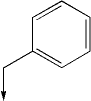 |
| 331332333 | rac+− | 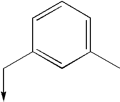 |
| 334335336 | rac+− | 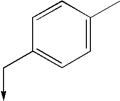 |

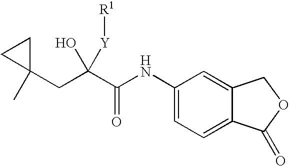

| | | |
|---|---|---|
| 337338339 | rac+− | 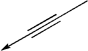 |
| 340341342 | rac+− | 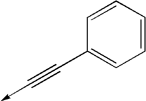 |
| 343344345 | rac+− | 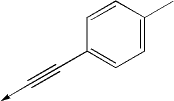 |
| 346347348 | rac+− | 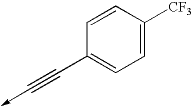 |
| 349350351 | rac+− | 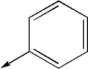 |
| 352353354 | rac+− | 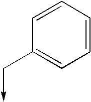 |
| 355356357 | rac+− | 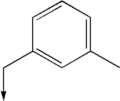 |
| 358359360 | rac+− | 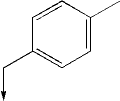 |

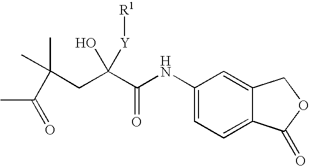

| | | |
|---|---|---|
| 361362363 | rac+− | 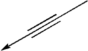 |
| 364365366 | rac+− | 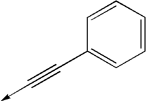 |
| 367368369 | rac+− | 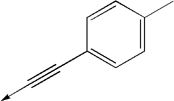 |
| 370371372 | rac+− | 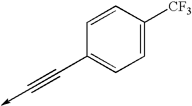 |
| 373374375 | rac+− | 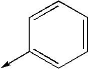 |
| 376377378 | rac+− | 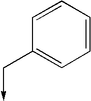 |
| 379380381 | rac+− | 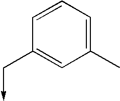 |
| 382383384 | rac+− | 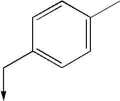 |

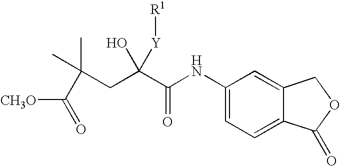

| | | |
|---|---|---|
| 385386387 | rac+− | 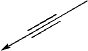 |
| 388389390 | rac+− | 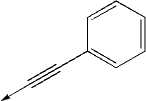 |
| 391392393 | rac+− | 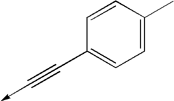 |
| 394395396 | rac+− | 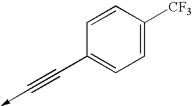 |
| 397398399 | rac+− | 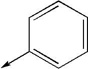 |
| 400401402 | rac+− | 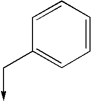 |
| 403404405 | rac+− | 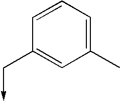 |
| 406407408 | rac+− | 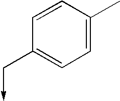 |

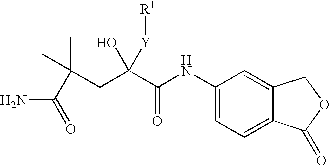

| | | |
|---|---|---|
| 409410411 | rac+− | 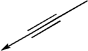 |
| 412413414 | rac+− | 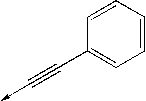 |
| 415416417 | rac+− | 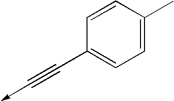 |
| 418419420 | rac+− | 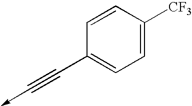 |
| 421422423 | rac+− | 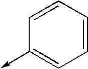 |
| 424425426 | rac+− | 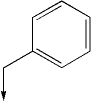 |
| 427428429 | rac+− | 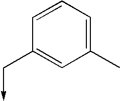 |
| 430431432 | rac+− | 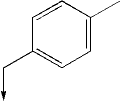 |

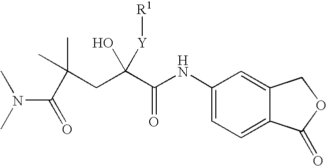

| | | |
|---|---|---|
| 433434435 | rac+− | 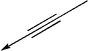 |
| 436437438 | rac+− | 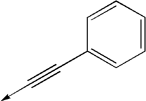 |
| 439440441 | rac+− | 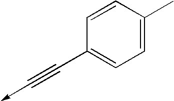 |
| 442443444 | rac+− | 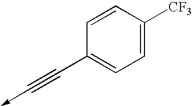 |
| 445446447 | rac+− | 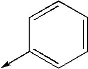 |
| 448449450 | rac+− | 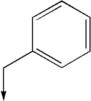 |
| 451452453 | rac+− | 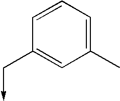 |
| 454455456 | rac+− | 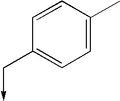 |

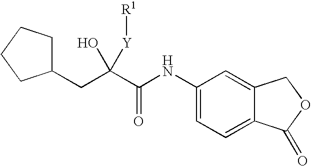

| | | |
|---|---|---|
| 457458459 | rac+− | 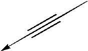 |
| 460461462 | rac+− | 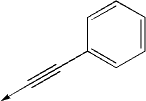 |
| 463464465 | rac+− | 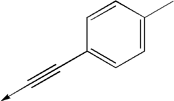 |
| 466467468 | rac+− | 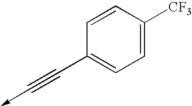 |
| 469470471 | rac+− | 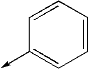 |
| 472473474 | rac+− | 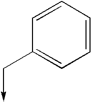 |
| 475476477 | rac+− | 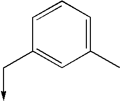 |
| 478479480 | rac+− | 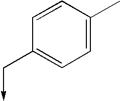 |

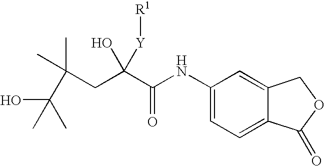

| | | |
|---|---|---|
| 481482483 | rac+− | 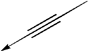 |
| 484485486 | rac+− | 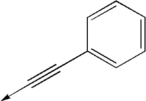 |
| 487488489 | rac+− | 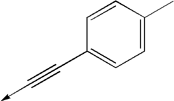 |
| 490491492 | rac+− | 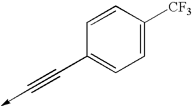 |
| 493494495 | rac+− | 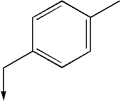 |
| 496497498 | rac+− | 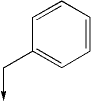 |
| 499500501 | rac+− | 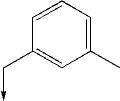 |
| 502503504 | rac+− | 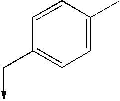 |

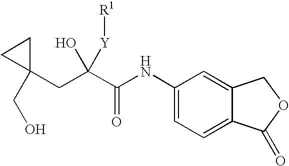

| | | |
|---|---|---|
| 505506507 | rac+− | 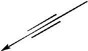 |
| 508509510 | rac+− | 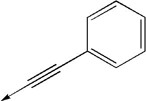 |
| 511512513 | rac+− | 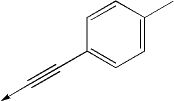 |
| 514515516 | rac+− | 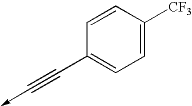 |
| 517518519 | rac+− | 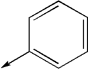 |
| 520521522 | rac+− | 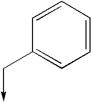 |
| 523524525 | rac+− | 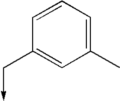 |
| 526527528 | rac+− | 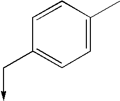 |

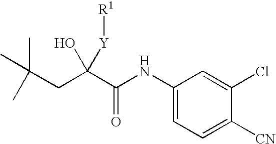

| | | |
|---|---|---|
| 529530531 | rac+− | 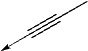 |
| 532533534 | rac+− | 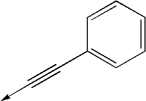 |
| 535536537 | rac+− | 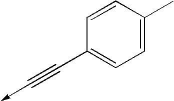 |
| 538539540 | rac+− | 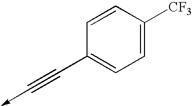 |
| 541542543 | rac+− | 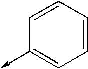 |
| 544545546 | rac+− | 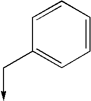 |
| 547548549 | rac+− | 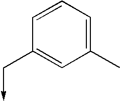 |
| 550551552 | rac+− | 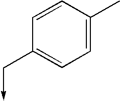 |

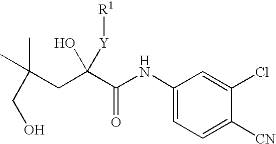

| | | |
|---|---|---|
| 553554555 | rac+− | 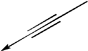 |
| 556557558 | rac+− | 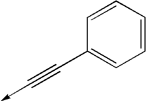 |
| 559560561 | rac+− | 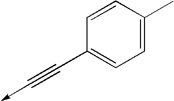 |
| 562563564 | rac+− | 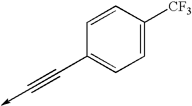 |
| 565566567 | rac+− | 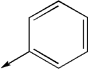 |
| 568569570 | rac+− | 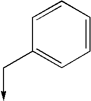 |
| 571572573 | rac+− | 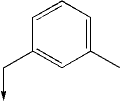 |
| 574575576 | rac+− | 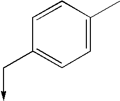 |

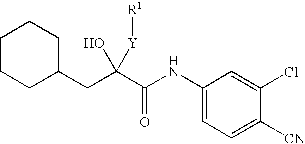

| | | |
|---|---|---|
| 577578579 | rac+− | 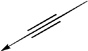 |
| 580581582 | rac+− | 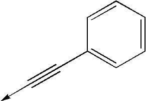 |
| 583584585 | rac+− | 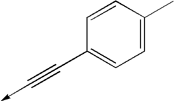 |
| 586587588 | rac+− | 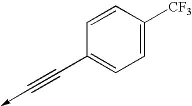 |
| 589590591 | rac+− | 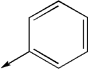 |
| 592593594 | rac+− | 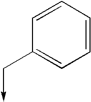 |
| 595596597 | rac+− | 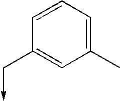 |
| 598599600 | rac+− | 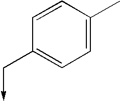 |

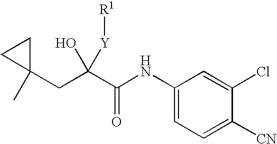

| | | |
|---|---|---|
| 601602603 | rac+− | 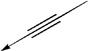 |
| 604605606 | rac+− | 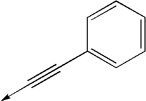 |
| 607608609 | rac+− | 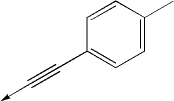 |
| 610611612 | rac+− | 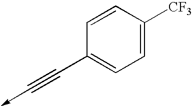 |
| 613614615 | rac+− | 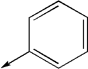 |
| 616617618 | rac+− | 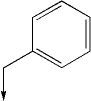 |
| 619620621 | rac+− | 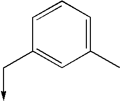 |
| 622623624 | rac+− | 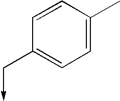 |

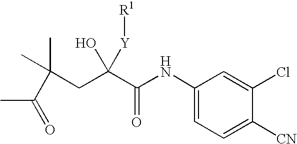

| | | |
|---|---|---|
| 625626627 | rac+− | 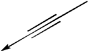 |
| 628629630 | rac+− | 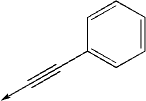 |
| 631632633 | rac+− | 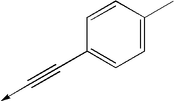 |
| 634635636 | rac+− | 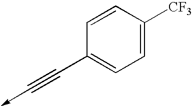 |
| 637638639 | rac+− | 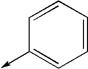 |
| 640641642 | rac+− | 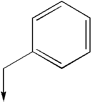 |
| 643644645 | rac+− | 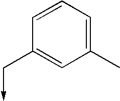 |
| 646647648 | rac+− | 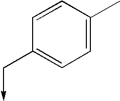 |

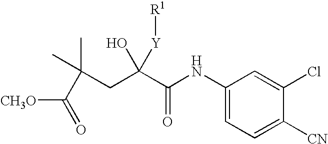

| | | |
|---|---|---|
| 649650651 | rac+− | 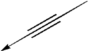 |
| 652653654 | rac+− | 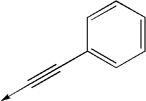 |
| 655656657 | rac+− | 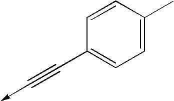 |
| 658659660 | rac+− | 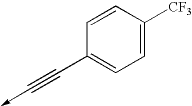 |
| 661662663 | rac+− | 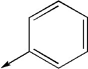 |
| 664665666 | rac+− | 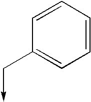 |
| 667668669 | rac+− | 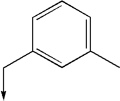 |
| 670671672 | rac+− | 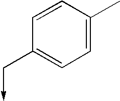 |

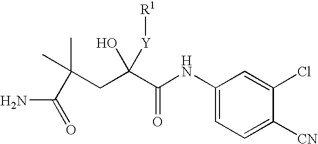

| | | |
|---|---|---|
| 673674675 | rac+− | 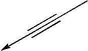 |
| 676677678 | rac+− | 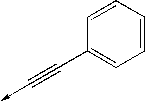 |
| 679680681 | rac+− | 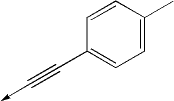 |
| 682683684 | rac+− | 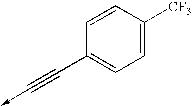 |
| 685686687 | rac+− | 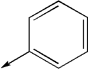 |
| 688689690 | rac+− | 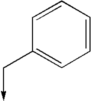 |
| 691692693 | rac+− | 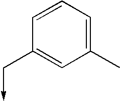 |
| 694695696 | rac+− | 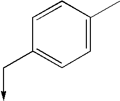 |

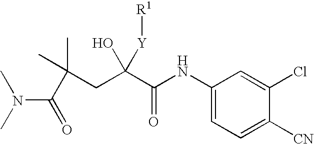

| | | |
|---|---|---|
| 697698699 | rac+− | 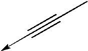 |
| 700701702 | rac+− | 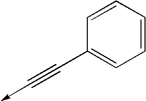 |
| 703704705 | rac+− | 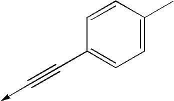 |
| 706707708 | rac+− | 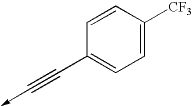 |
| 709710711 | rac+− | 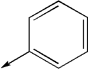 |
| 712713714 | rac+− | 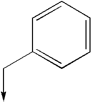 |
| 715716717 | rac+− | 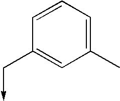 |
| 718719720 | rac+− | 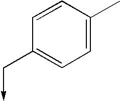 |

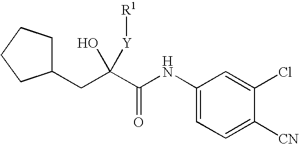

| | | |
|---|---|---|
| 721722723 | rac+− | 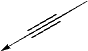 |
| 724725726 | rac+− | 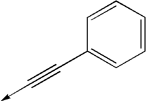 |
| 727728729 | rac+− | 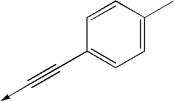 |
| 730731732 | rac+− | 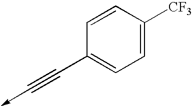 |
| 733734735 | rac+− | 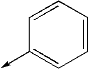 |
| 736737738 | rac+− | 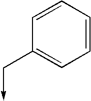 |
| 739740741 | rac+− | 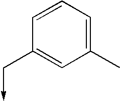 |
| 742743744 | rac+− | 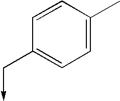 |

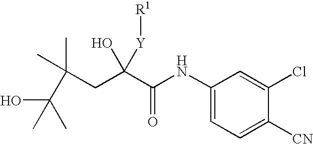

| | | |
|---|---|---|
| 745746747 | rac+− | 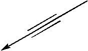 |
| 748749750 | rac+− | 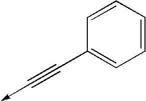 |
| 751752753 | rac+− | 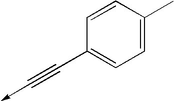 |
| 754755756 | rac+− | 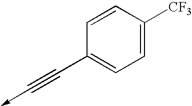 |
| 757758759 | rac+− | 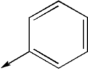 |
| 760761762 | rac+− | 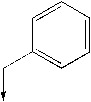 |
| 763764765 | rac+− | 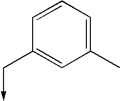 |
| 766767768 | rac+− | 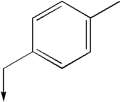 |

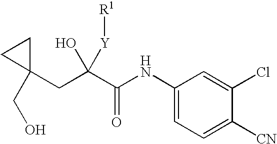

| | | |
|---|---|---|
| 769770771 | rac+− | 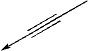 |
| 772773774 | rac+− | 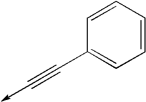 |
| 775776777 | rac+− | 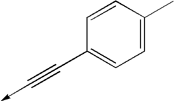 |
| 778779780 | rac+− | 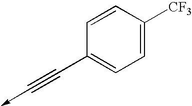 |
| 781782783 | rac+− | 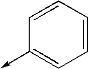 |
| 784785786 | rac+− | 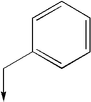 |
| 787788789 | rac+− | 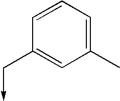 |
| 790791792 | rac+− | 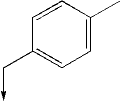 |

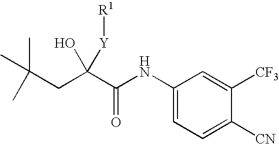

| | | |
|---|---|---|
| 793794795 | rac+− | 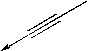 |
| 796797798 | rac+− | 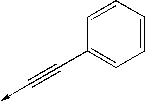 |
| 799800801 | rac+− | 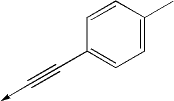 |
| 802803804 | rac+− | 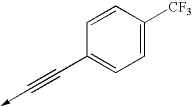 |
| 805806807 | rac+− | 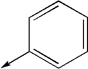 |
| 808809810 | rac+− | 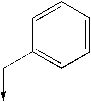 |
| 811812813 | rac+− | 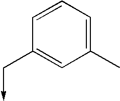 |
| 814815816 | rac+− | 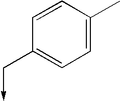 |

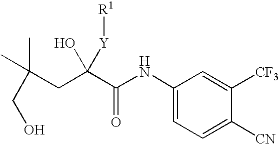

| | | |
|---|---|---|
| 817818819 | rac+− | 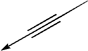 |
| 820821822 | rac+− | 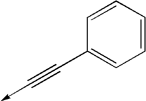 |
| 823824825 | rac+− | 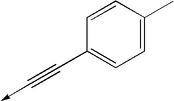 |
| 826827828 | rac+− | 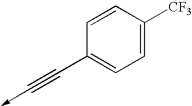 |
| 829830831 | rac+− | 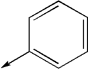 |
| 832833834 | rac+− | 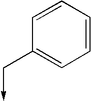 |
| 835836837 | rac+− | 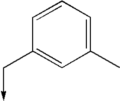 |
| 838839840 | rac+− | 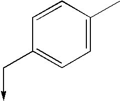 |

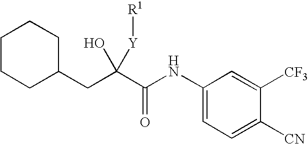

| | | |
|---|---|---|
| 841842843 | rac+− | 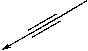 |
| 844845846 | rac+− | 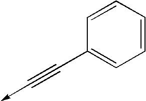 |
| 847848849 | rac+− | 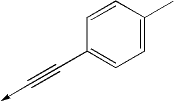 |
| 850851852 | rac+− | 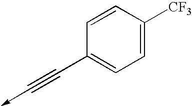 |
| 853854855 | rac+− | 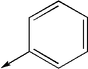 |
| 856857858 | rac+− | 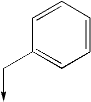 |
| 859860861 | rac+− | 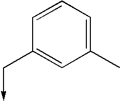 |
| 862862863 | rac+− | 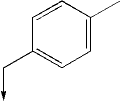 |

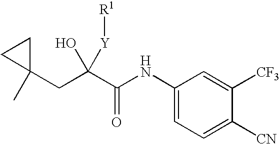

| | | |
|---|---|---|
| 865866867 | rac+− | 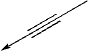 |
| 868869870 | rac+− | 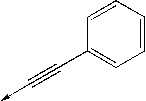 |
| 871872873 | rac+− | 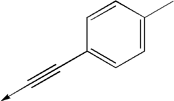 |
| 874875876 | rac+− | 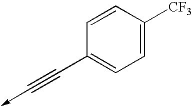 |
| 877878879 | rac+− | 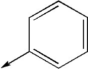 |
| 880881882 | rac+− | 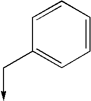 |
| 883884885 | rac+− | 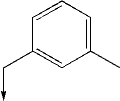 |
| 886887888 | rac+− | 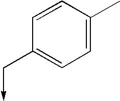 |

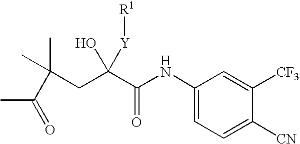

| | | |
|---|---|---|
| 889890891 | rac+− | 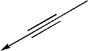 |
| 892893894 | rac+− | 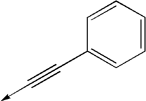 |
| 895896897 | rac+− | 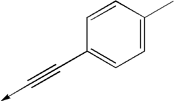 |
| 898899900 | rac+− | 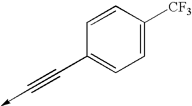 |
| 901902903 | rac+− | 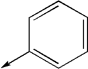 |
| 904905906 | rac+− | 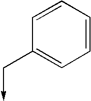 |
| 907908909 | rac+− | 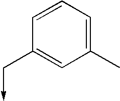 |
| 910911912 | rac+− | 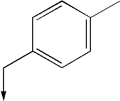 |

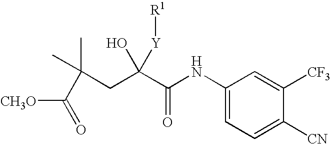

| | | |
|---|---|---|
| 913914915 | rac+− | 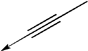 |
| 916917918 | rac+− | 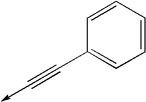 |
| 919920921 | rac+− | 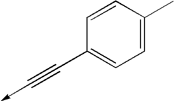 |
| 922923924 | rac+− | 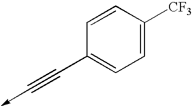 |
| 925926927 | rac+− | 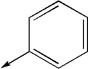 |
| 928929930 | rac+− | 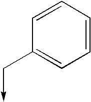 |
| 931932933 | rac+− | 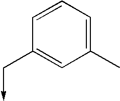 |
| 934935936 | rac+− | 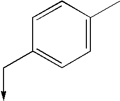 |

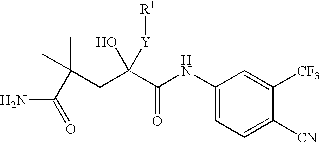

| | | |
|---|---|---|
| 937938939 | rac+− | 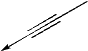 |
| 940941942 | rac+− | 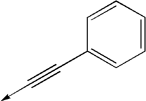 |
| 943944945 | rac+− | 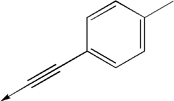 |
| 946947948 | rac+− | 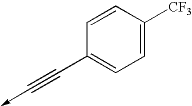 |
| 949950951 | rac+− | 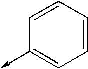 |
| 952953954 | rac+− | 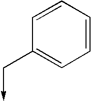 |
| 955956957 | rac+− | 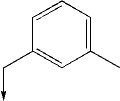 |
| 958959960 | rac+− | 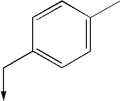 |

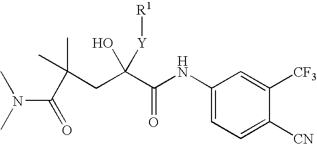

| | | |
|---|---|---|
| 961962963 | rac+− | 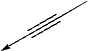 |
| 964965966 | rac+− | 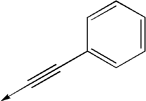 |
| 967968969 | rac+− | 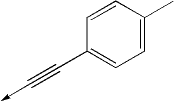 |
| 970971972 | rac+− | 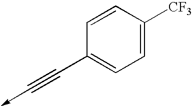 |
| 973974975 | rac+− | 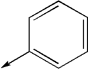 |
| 976977978 | rac+− | 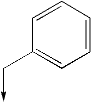 |
| 979980981 | rac+− | 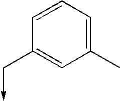 |
| 982983984 | rac+− | 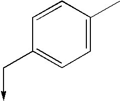 |

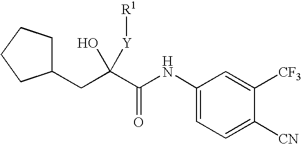

| | | |
|---|---|---|
| 985986987 | rac+− | 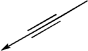 |
| 988989990 | rac+− | 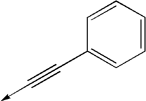 |
| 991992993 | rac+− | 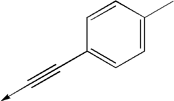 |
| 994995996 | rac+− | 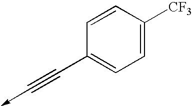 |
| 997998999 | rac+− | 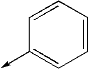 |
| 100010011002 | rac+− | 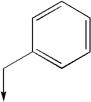 |
| 100310041005 | rac+− | 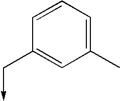 |
| 100610071008 | rac+− | 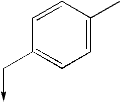 |

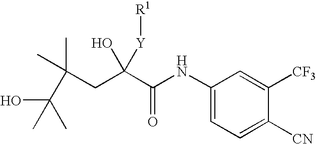

| | | |
|---|---|---|
| 100910101011 | rac+− | 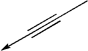 |
| 101210131014 | rac+− | 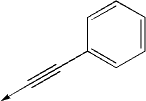 |
| 101510161017 | rac+− | 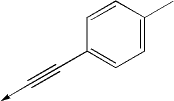 |
| 101810191020 | rac+− | 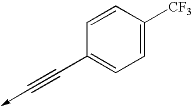 |
| 102110221023 | rac+− | 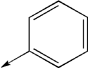 |
| 102410251026 | rac+− | 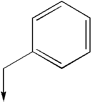 |
| 102710281029 | rac+− | 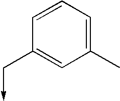 |
| 103010311032 | rac+− | 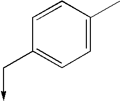 |

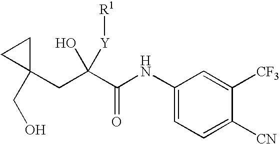

| | | |
|---|---|---|
| 103310341035 | rac+− | 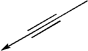 |
| 103610371038 | rac+− | 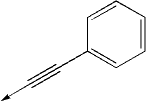 |
| 103910401041 | rac+− | 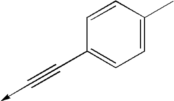 |
| 104210431044 | rac+− | 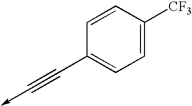 |
| 104510461047 | rac+− | 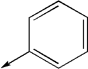 |
| 104810491050 | rac+− | 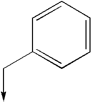 |
| 105110521053 | rac+− | 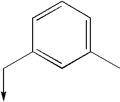 |
| 105410551056 | rac+− | 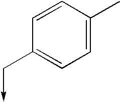 |

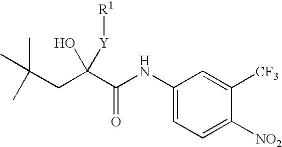

| | | |
|---|---|---|
| 105710581059 | rac+− | 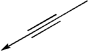 |
| 106010611062 | rac+− | 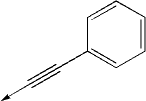 |
| 106310641065 | rac+− | 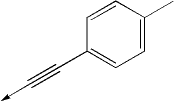 |
| 106610671068 | rac+− | 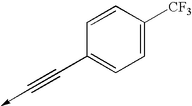 |
| 106910701071 | rac+− | 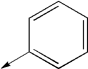 |
| 107210731074 | rac+− | 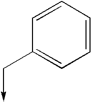 |
| 107510761077 | rac+− | 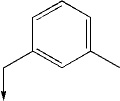 |
| 107810791080 | rac+− | 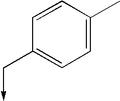 |

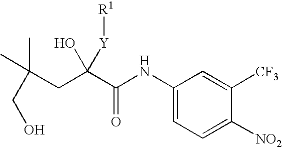

| | | |
|---|---|---|
| 108110821083 | rac+− | 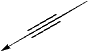 |
| 108410851086 | rac+− | 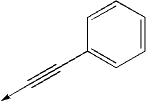 |
| 108710881089 | rac+− | 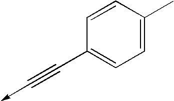 |
| 109010911092 | rac+− | 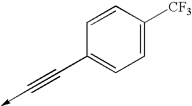 |
| 109310941095 | rac+− | 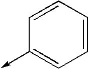 |
| 109610971098 | rac+− | 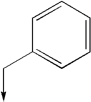 |
| 109911001101 | rac+− | 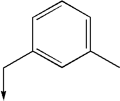 |
| 110211031104 | rac+− | 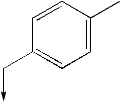 |

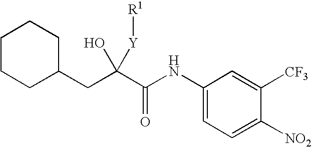

| | | |
|---|---|---|
| 110511061107 | rac+− | 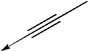 |
| 110811091110 | rac+− | 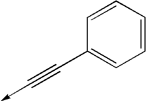 |
| 111111121113 | rac+− | 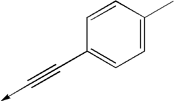 |
| 111411151116 | rac+− | 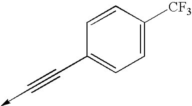 |
| 111711181119 | rac+− | 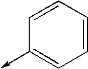 |
| 112011211122 | rac+− | 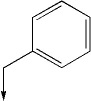 |
| 112311241125 | rac+− | 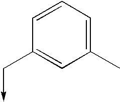 |
| 112611271128 | rac+− | 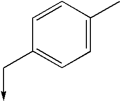 |

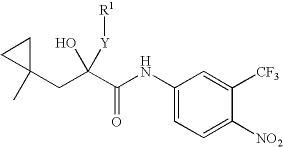

| | | |
|---|---|---|
| 112911301131 | rac+− | 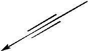 |
| 113211331134 | rac+− | 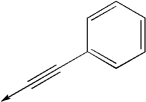 |
| 113511361137 | rac+− | 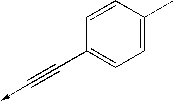 |
| 113811391140 | rac+− | 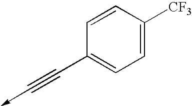 |
| 114111421143 | rac+− | 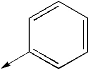 |
| 114411451146 | rac+− | 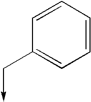 |
| 114711481149 | rac++ | 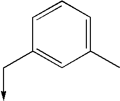 |
| 115011511152 | rac+− | 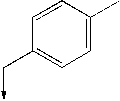 |

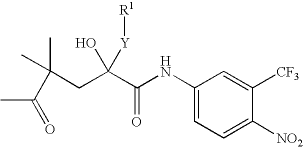

| | | |
|---|---|---|
| 115311541155 | rac+− | 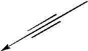 |
| 115611571158 | rac+− | 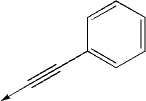 |
| 115911601161 | rac+− | 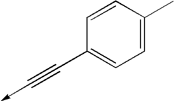 |
| 116211631164 | rac+− | 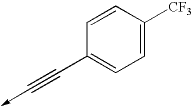 |
| 116511661167 | rac+− | 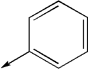 |
| 116811691170 | rac+− | 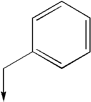 |
| 117111721173 | rac+− | 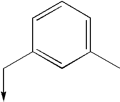 |
| 117411751176 | rac+− | 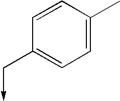 |

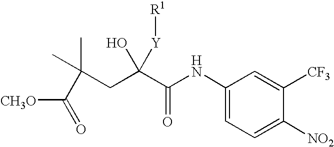

| | | |
|---|---|---|
| 117711781179 | rac+− | 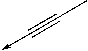 |
| 118011811182 | rac+− | 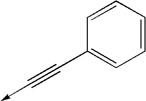 |
| 118311841185 | rac+− | 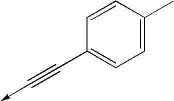 |
| 118611871188 | rac+− | 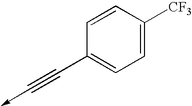 |
| 118911901191 | rac+− | 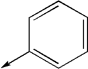 |
| 119211931194 | rac+− | 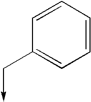 |
| 119511961197 | rac+− | 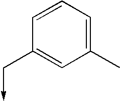 |
| 119811991200 | rac+− | 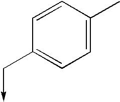 |

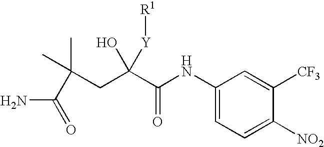

| | | |
|---|---|---|
| 120112021203 | rac+− | 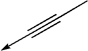 |
| 120412051206 | rac+− | 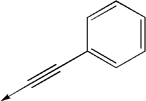 |
| 120712081209 | rac+− | 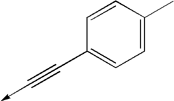 |
| 121012111212 | rac+− | 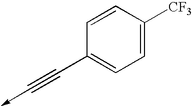 |
| 121312141215 | rac+− | 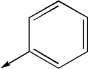 |
| 121612171218 | rac+− | 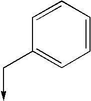 |
| 121912201221 | rac+− | 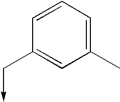 |
| 122212231224 | rac+− | 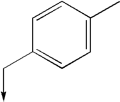 |

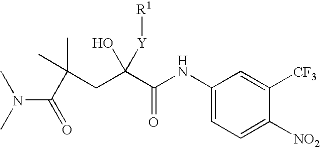

| | | |
|---|---|---|
| 122512261227 | rac+− | 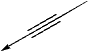 |
| 122812291230 | rac+− | 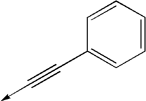 |
| 123112321233 | rac+− | 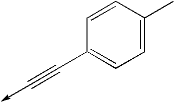 |
| 123412351236 | rac+− | 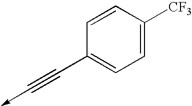 |
| 123712381239 | rac+− | 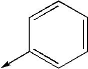 |
| 124012511242 | rac+− | 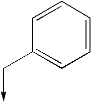 |
| 124312441245 | rac+− | 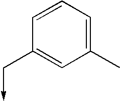 |
| 124612471248 | rac+− | 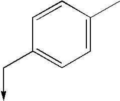 |

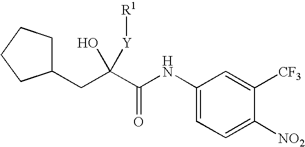

| | | |
|---|---|---|
| 124912501251 | rac+− | 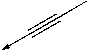 |
| 125212531254 | rac+− | 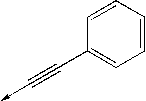 |
| 125512561257 | rac+− | 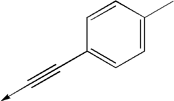 |
| 125812591260 | rac+− | 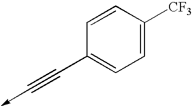 |
| 126112621263 | rac+− | 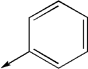 |
| 126412651266 | rac+− | 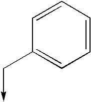 |
| 126712681269 | rac+− | 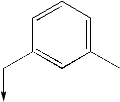 |
| 127012711272 | rac+− | 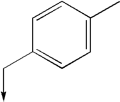 |

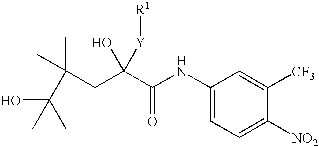

| | | |
|---|---|---|
| 127312741275 | rac+− | 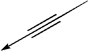 |
| 127612771278 | rac+− | 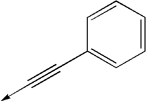 |
| 127912801281 | rac+− | 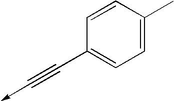 |
| 128212831284 | rac+− | 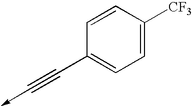 |
| 128512861287 | rac+− | 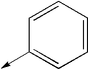 |
| 128812891290 | rac+− | 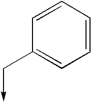 |
| 129112921293 | rac+− | 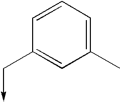 |
| 129412951296 | rac+− | 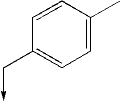 |

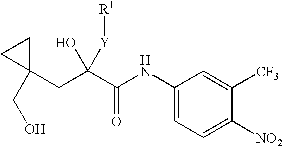

| | | |
|---|---|---|
| 129712981299 | rac+− | 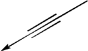 |
| 130013011302 | rac+− | 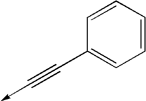 |
| 130313041305 | rac+− | 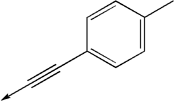 |
| 130613071308 | rac+− | 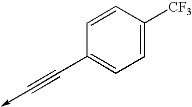 |
| 130913101311 | rac+− | 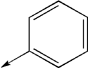 |
| 131213131314 | rac+− | 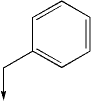 |
| 131513161317 | rac+− | 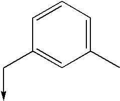 |
| 131813191320 | rac+− | 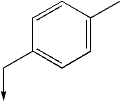 |

Biological Characterization of the Compounds According to the Invention

Progesterone receptor modulators can be identified with the aid of simple methods, test programmes known to the skilled person. It is possible for this purpose for example to incubate a compound to be tested together with a progestogen in a test system for progesterone receptor ligands and to check whether the effect mediated by progesterone is altered in the presence of the modulator in this test system.

The substances according to the invention of the general formula I were tested in the following models:

Progesterone Receptor-Binding Assay

Measurement of the receptor binding affinity:

The receptor binding affinity was determined by competitive binding of a specifically binding ³H-labelled hormone (tracer) and of the compound to be tested on receptors in the cytosol from animal target organs. The aim in this case was receptor saturation and reaction equilibrium.

The tracer and increasing concentrations of the compound to be tested (competitor) were coincubated at 0-4° C. for 18 h with the receptor-containing cytosol fraction. After removal of unbound tracer with carbon-dextran suspension, the receptor-bound tracer content was measured for each concentration, and the IC₅₀ was determined from the concentration series. The relative molar binding affinity (RBA) was calculated as ratio of the IC₅₀ values for reference substance and compound to be tested (×100%) (RBA of the reference substance =100%).

The following incubation conditions were chosen for the receptor types:

Progesterone receptor:

Uterus cytosol of the estradiol-primed rabbit, homogenized in TED buffer (20 mMTris/HCl, pH 7.4; 1 mM ethylenediamine tetraacetate, 2 mM dithiothreitol) with 250 mM sucrose; stored at −30° C. Tracer: ³H-ORG 2058, 5 nM; reference substance: progesterone.

Glucocorticoid receptor:

Thymus cytosol from the adrenalectomized rat, thymi stored at −30° C.; buffer: TED. Tracer: ³H-dexamethasone, 20 nM; reference substance: dexamethasone.

The competition factors (CF values) for the compounds according to the invention of the general formula (I) on the progesterone receptor are between 0.2 and 35 relative to progesterone. The CF values on the glucocorticoid receptor are in the range from 3 to 35 relative to dexamethasone.

The compounds according to the invention accordingly have a high affinity for the progesterone receptor, but only a low affinity for the glucocorticoid receptor.

Antagonism on the Progesterone Receptor PR

The transactivation assay is carried out as described in WO 02/054064.

The IC₅₀ values are in the range from 0.1 to 150 nM.

Agonism on the Progesterone Receptor PR

The transactivation assay is carried out as described in Fuhrmann et al. (Fuhrmann U., Hess-Stump H., Cleve A., Neef G., Schwede W., Hoffmann J., Fritzemeier K.-H., Chwalisz K., Journal of Medicinal Chemistry, 43, 26, 2000, 5010-5016). The EC₅₀ values are in the range from 0.01 to 150 nM.

Dosage

The progesterone receptor modulators can be administered orally, enterally, parenterally or transdermally for the use according to the invention.

Satisfactory results are generally to be expected in the treatment of the indications mentioned hereinbefore when the daily doses cover a range from 1 μg to 1000 mg of the compound according to the invention for gynaecological indications such as the treatment of endometriosis, leiomyomas of the uterus and dysfunctional bleeding, and for use in fertility control and for hormone replacement therapy. For oncological indications, daily dosages in the range from 1 μg to 2000 mg of the compound according to the invention are to be administered.

Suitable dosages of the compounds according to the invention in humans for the treatment of endometriosis, of leiomyomas of the uterus and dysfunctional bleeding and for use in fertility control and for hormone replacement therapy are from 50 μg to 500 mg per day, depending on the age and constitution of the patient, it being possible to administer the necessary daily dose by single or multiple administration.

The dosage range for the compounds according to the invention for the treatment of breast carcinomas is 10 mg to 2000 mg per day.

The pharmaceutical products based on the novel compounds are formulated in a manner known per se by processing the active ingredient with the carrier substances, fillers, substances influencing disintegration, binders, humectants, lubricants, absorbents, diluents, masking flavours, colorants, etc. which are used in pharmaceutical technology, and converting into the desired administration form. Reference should be made in this connection to Remington's Pharmaceutical Science, 15^{th} ed. Mack Publishing Company, East Pennsylvania (1980).

Suitable for oral administration are in particular tablets, film-coated tablets, sugar-coated tablets, capsules, pills, powders, granules, pastilles, suspensions, emulsions or solutions.

Preparations for injection and infusion are possible for parenteral administration.

Appropriately prepared crystal suspensions can be used for intraarticular injection.

Aqueous and oily solutions for injection or suspensions and corresponding depot preparations can be used for intramuscular injection.

For rectal administration, the novel compounds can be used in the form of suppositories, capsules, solutions (e.g. in the form of enemas) and ointments, both for systemic and for local therapy.

Furthermore, compositions for vaginal use may also be mentioned as preparation.

For pulmonary administration of the novel compounds, they can be used in the form of aerosols and inhalants.

Patches are possible for transdermal administration, and formulations in gels, ointments, fatty ointments, creams, pastes, dusting powders, milk and tinctures are possible for topical application. The dosage of the compounds of the general formula I in these preparations should be 0.01%-20% in order to achieve an adequate pharmacological effect.

Corresponding tablets can be obtained for example by mixing the active ingredient with known excipients, for example inert diluents such as dextrose, sugar, sorbitol, mannitol, polyvinylpyrrolidone, disintegrants such as maize starch or alginic acid, binders such as starch or gelatin, lubricants such as magnesium stearate or talc and/or means to achieve a depot effect such as carboxypolymethylene, carboxymethylcellulose, cellulose acetate phthalate or polyvinyl acetate. The tablets may also consist of a plurality of layers.

Correspondingly, coated tablets can be produced by coating cores produced in analogy to the tablets with compositions normally used in tablet coatings, for example polyvinylpyrrolidone or shellac, gum arabic, talc, titanium oxide or sugar. The tablet covering may in this case also consist of a plurality of layers, it being possible to use the excipients mentioned above for tablets.

Solutions or suspensions of the compounds according to the invention of the general formula I may additionally comprise taste-improving agents such as saccharin, cyclamate or sugar, and, for example, flavourings such as vanillin or orange extract. They may additionally comprise suspending excipients such as sodium carboxymethylcellulose or preservatives such as p-hydroxybenzoates.

Capsules comprising the compounds of the general formula I can be produced for example by mixing the compound(s) of the general formula I with an inert carrier such as lactose or sorbitol and encapsulating it in gelatin capsules.

Suitable suppositories can be produced for example by mixing with carriers intended for this purpose, such as neutral fats or polyethylene glycol or derivatives thereof.

The compounds according to the invention of the general formula (I) or their pharmaceutically acceptable salts can be used, because of their antagonistic or partial agonistic activity, for the manufacture of a medicament, in particular for the treatment and prophylaxis of gynaecological disorders such as endometriosis, leiomyomas of the uterus, dysfunctional bleeding and dysmenorrhoea. They can furthermore be employed to counteract hormonal irregularities, for inducing menstruation and alone or in combination with prostaglandins and/or oxytocin to induce labour.

The compounds according to the invention of the general formula (I) or their pharmaceutically acceptable salts are furthermore suitable for the manufacture of products for female contraception (see also WO 93/23020, WO 93/21927).

The compounds according to the invention or their pharmaceutically acceptable salts can additionally be employed alone or in combination with a selective estrogen receptor modulator (SERM) for female hormone replacement therapy.

In addition, the said compounds have an antiproliferative effect in hormone-dependent tumours. They are therefore suitable for the therapy of hormone-dependent carcinomas such as, for example, for breast, prostate and endometrial carcinomas.

The compounds according to the invention or their pharmaceutically acceptable salts can be employed for the treatment of hormone-dependent carcinomas both in first-line therapy and in second-line therapy, especially after tamoxifen failure.

The compounds according to the invention, having antagonistic or partially agonistic activity, of the general formula (I) or their pharmaceutically acceptable salts can also be used in combination with compounds having antiestrogenic activity (estrogen receptor antagonists or aromatase inhibitors) or selective estrogen receptor modulators (SERM) for producing pharmaceutical products for the treatment of hormone-dependent tumours. The compounds according to the invention can likewise be used in combination with SERMs or an antiestrogen (estrogen receptor antagonist or aromatase inhibitor) for the treatment of endometriosis or of leiomyomas of the uterus.

Suitable for combination with the non-steroidal progesterone receptor modulators according to the invention in this connection are for example the following antiestrogens (estrogen receptor antagonists or aromatase inhibitors) or SERMs: tamoxifen, 5-(4-{5-[(RS)-(4,4,5,5,5-pentafluoropentyl)sulphinyl]pentyloxy}phenyl)-6-phenyl-8,9-dihydro-7H-benzocyclohepten-2-ol (WO 00/03979), ICI 182 780 (7alpha-[9-(4,4,5,5-pentafluoropentylsulphinyl)nonyl]estra-1,3,5(10)-triene-3,17beta-diol), 11beta-fluoro-7alpha-[5-(methyl{3-[(4,4,5,5,5-pentafluoropentyl)sulphanyl]propyl}amino)pentyl]-estra-1,3,5(10)-triene-3,17beta-diol (WO98/07740), 11beta-fluoro-7alpha-{5-[methyl(7,7,8,8,9,9,10,10,10-nonafluorodecyl)amino]pentyl}estra-1,3,5(10)-triene-3,17-beta-diol (WO 99/33855), 11beta-fluoro-17alpha-methyl-7alpha-{5-[methyl(8,8,9,9,9-pentafluorononyl)amino]pentyl}estra-1,3,5(10)-triene-3,17beta-diol (WO 03/045972), clomifene, raloxifene, and further compounds having antiestrogenic activity, and aromatase inhibitors such as, for example, fadrozole, formestane, letrozole, anastrozole or atamestane.

Finally, the present invention also relates to the use of the compounds of the general formula I, where appropriate together with an antiestrogen or SERM, for the manufacture of a medicament.

The present invention further relates to pharmaceutical compositions which comprise at least one compound according to the invention, where appropriate in the form of a pharmaceutically/pharmacologically acceptable salt.

These pharmaceutical compositions and medicaments may be intended for oral, rectal, vaginal, subcutaneous, percutaneous, intravenous or intramuscular administration. Besides conventional carriers and/or diluents, they comprise at least one compound according to the invention.

The medicaments of the invention are manufactured with the conventional solid or liquid carriers or diluents and the excipients normally used in pharmaceutical technology appropriate for the desired mode of administration with a suitable dosage in a known manner. The preferred preparations consist of a dosage form suitable for oral administration. Examples of such dosage forms are tablets, film-coated tablets, sugar-coated tablets, capsules, pills, powders, solutions or suspensions, where appropriate as depot form.

The pharmaceutical compositions comprising at least one of the compounds according to the invention are preferably administered orally.

Also suitable are parenteral preparations such as solutions for injection. Further preparations which may also be mentioned are for example suppositories and compositions for vaginal use.

Preparation of the Inventive Compounds:

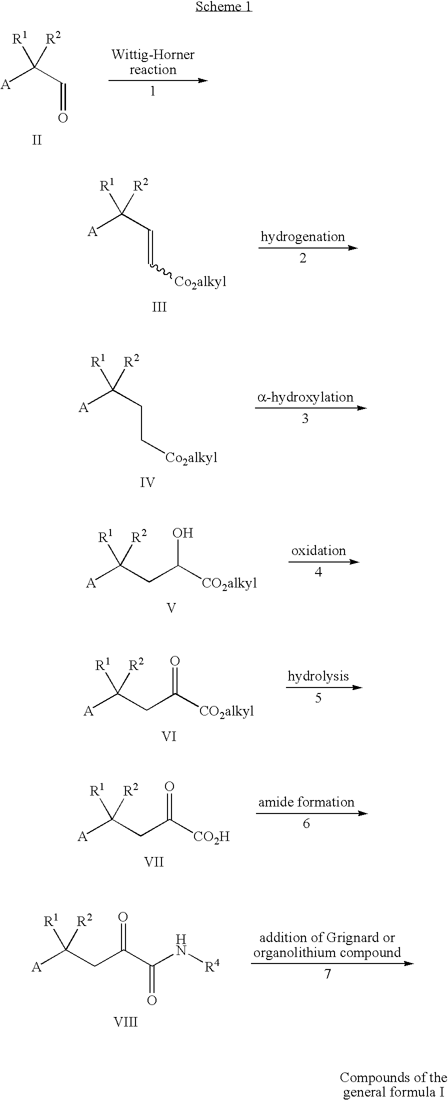

The compounds of the general formula I may be synthesized, for example, as shown in scheme 1. Chain extension proceeding from aldehydes of type II can be effected, for example, by means of Horner-Wittig reaction. Reduction of the double bond, for example by hydrogenation in the presence of suitable catalysts, then gives rise to compounds of the general formula IV, which give rise to compounds of the general formula VI by an α-hydroxylation followed by an oxidation of the alcohol formed to the ketone. For the α-hydroxylation to prepare compounds of the general formula V, various processes known from the literature are useful, for example the use of 2-sulphonyloxaziridine by a process described by Davis et al. (J. Org. Chem, 1984, 49, 3241). The oxidation to compounds of the general formula VI can then be effected by known standard methods. The amides of the general formula VIII are prepared, for example, via the formation of the acid chlorides and subsequent reaction with the corresponding amines. Alternatively, for this purpose, it is also possible to utilize other methods of amide formation according to the amine to be introduced. The compounds of the general formula I are then prepared from the amides of the general formula VIII by repeated addition of Grignard or organolithium compounds. Steps 1-7 can, though, also be performed in an altered sequence.

In numerous cases, intermediate compounds of the general formulae III-VII are also commercially available.

The substituents A, X, Y, R¹, R², R³ and R⁴ may optionally be modified further after they have been introduced. Possible reactions for this purpose include, for example, oxidation, reduction, alkylations, acylations, nucleophilic additions or else transition metal-catalysed coupling reactions.

Functional groups in compounds of the general formulae II-VIII are optionally provided intermediately with protective groups which are then detached again at a suitable stage.

The examples which follow serve to illustrate the subject-matter of the invention in detail without any intention to restrict it to them.

The preparation of 6-amino-4-methyl-2,3-benzoxazin-1-one is described in WO 199854159.

### Preparation of 6-(4,4-dimethyl-2-oxovaleroylamino)-4-methyl-2,3-benzoxazin-1-one

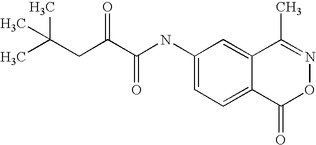

4,4-Dimethyl-2-oxopentanoic acid (0.75 g) was dissolved in 10 ml of N,N-dimethylacetamide. 460 μl of thionyl chloride were added at −10° C. and the mixture was left to stir at −10° C. for one hour. Subsequently, 1.28 g of 6-amino-4-methyl-2,3-benzoxazin-1-one were added in portions. The mixture was then stirred for a further 3 hours (−10° C. to 0° C). Subsequently, the reaction mixture was poured onto ice-water. It was extracted with ethyl acetate. The organic phase was washed with saturated aqueous sodium chloride solution, dried over sodium sulphate and concentrated under reduced pressure. The crude product was purified by column chromatography on silica gel with a mixture of hexane/ethyl acetate. 1.41 g of product were obtained.

¹H NMR (ppm, CDCl₃, 300 MHz): 1.08 (9H), 2.60 (3H), 2.93 (2H), 7.84 (1H), 8.31 (1H), 8.38 (1H); 9.18 (1H).

### EXAMPLE 1

### rac-6-[2(2,2-Dimethylpropyl)-2-hydroxypent-3-ynoylamino]-4-methyl-2,3-benzoxazin-1-one

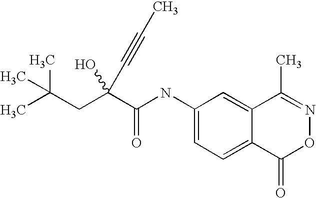

1-Propynylmagnesium bromide (2.65 ml, 0.5 M in tetrahydrofuran) was added to a solution, cooled to −70° C., of 6-(4,4-dimethyl-2-oxovaleroylamino)-4-methyl-2,3-benzoxazin-1-one (200 mg) in THF (5 ml). The reaction solution was allowed to come to room temperature under argon within 3 h and then stirred for a further 16 hours. Thereafter, the reaction mixture was poured onto ice-cold saturated ammonium chloride solution. It was extracted with ethyl acetate. The combined organic phases were washed with saturated sodium chloride solution and dried over sodium sulphate. The resulting crude product was chromatographed on silica gel. 168 mg of product were obtained.

¹H NMR (ppm, CDCl₃, 400 MHz): 1.08 (9H), 1.99 (3H), 2.07 (2H), 2.58 (3H), 2.90 (1H), 7.70 (1H), 7.83 (1H), 8.40 (1H), 9.10 (1H).

### EXAMPLE 2

### rac-6-[2(2,2-Dimethylpropyl)-2-hydroxy-4-phenylbut-3-ynoylamino]-4-methyl-2,3-benzoxazin-1-one

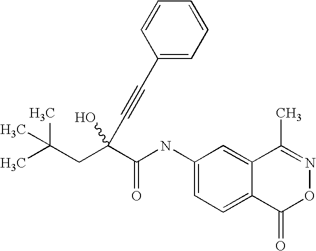

To a solution of 145 μl of phenylacetylene in tetrahydrofuran was added, at −78° C., n-butyllithium (830 μl, 1.6 M in hexane). The mixture was left to stir at this temperature for 30 minutes and then a solution of 6-(4,4-dimethyl-2-oxovaleroylamino)-4-methyl-2,3-benzoxazin-1-one (200 mg) in 5 ml tetrahydrofuran was added dropwise. Subsequently, the mixture was allowed to come to 23° C. over approx. 3 h and then stirred for 10 h. Thereafter, the reaction mixture was poured onto ice-cold saturated ammonium chloride solution. It was extracted with ethyl acetate. The combined organic phases were washed with saturated sodium chloride solution and dried over sodium sulphate. The crude product was chromatographed on silica gel. 199 mg of product were obtained.

¹H NMR (ppm, CDCl₃, 400 MHz): 1.14 (9H), 2.15-2.25 (2H), 2.58 (3H), 3.20 (1H), 7.27-7.49 (3H), 7.43 (2H), 7.22 (1H), 8.34 (1H), 8.40 (1H), 9.18 (1H).

### EXAMPLE 2a AND 2b

### (+)-6-[2(2,2-Dimethylpropyl)-2-hydroxy-4-phenylbut-3-ynoylamino]-4-methyl-2,3-benzoxazin-1-one 2a and (−)-6-[2(2,2-dimethylpropyl)-2-hydroxy-4-phenylbut-3-ynoylamino]-4-methyl-2,3-benzoxazin-1-one 2b

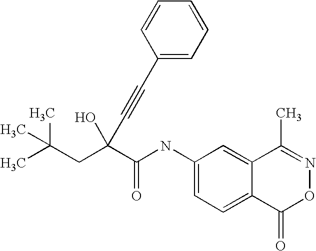

The racemic mixture obtained under Example 2 was separated by preparative chiral HPLC (column: Chiralpak AD 250×10 mm) into enantiomers 2a and 2b.

2a: [α]^{D}₂₀: +21.5° (CHCl₃, 10.3 mg/1 ml; λ=589 nm)

2b: [α]^{D}₂₀: −21.9° (CHCl₃, 10.4 mg/1 ml; λ=589 nm)

Examples 3 and 4 were synthesized to Example 2:

### EXAMPLE 3

### rac-6-[2(2,2-Dimethylpropyl)-2-hydroxy-4-(4-methylphenyl)but-3-ynoylamino]-4-methyl-2,3-benzoxazin-1-one

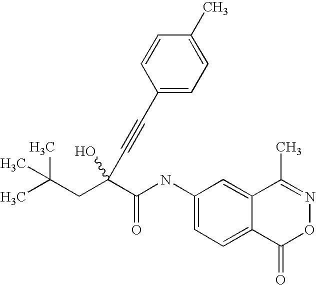

¹H NMR (ppm, CDCl₃, 400 MHz): 1.13 (9H), 2.12-2.25 (2H), 2.32 (3H), 2.56 (3H), 3.39 (1H), 7.10 (2H), 7.30 (2H), 7.73 (1H), 8.31 (1H), 8.38 (1H), 9.22 (1H).

### EXAMPLE 3a AND 3b

### (+)-6-[2(2,2-Dimethylpropyl)-2-hydroxy-4-(4-methylphenyl)but-3-ynoylamino]-4-methyl-2,3-benzoxazin-1-one 3a and (−)-6-[2(2,2-dimethylpropyl)-2-hydroxy-4-(4-methylphenyl)but-3-ynoylamino]-4-methyl-2,3-benzoxazin-1-one 3b

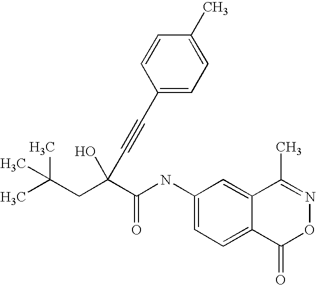

The racemic mixture obtained under Example 3 was separated by preparative chiral HPLC (column: Chiralpak AD 250×10 mm) into enantiomers 3a and 3b.

3a: [α]^{D}₂₀: +24.80 (CHCl₃, 11.2 mg/1 ml; λ=589 nm)

3b: [α]^{D}₂₀: −19.2° (CHCl₃, 5.1 mg/1 ml; λ=589 nm)

### EXAMPLE 4

### rac-6-[2(2,2-Dimethylpropyl)-2-hydroxy-4-(4-trifluoromethylphenyl)but-3-ynoylamino]-4-methyl-2,3-benzoxazin-1-one

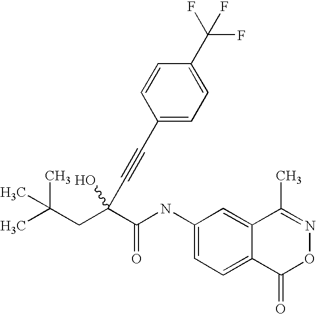

¹H NMR (ppm, CDCl₃, 400 MHz): 1.16 (9H), 2.16-2.28 (2H), 2.59 (3H), 3.21 (1H), 7.50-7.62 (4H), 7.76 (1H), 8.35 (1H), 8.40 (1H), 9.17 (1H).

### EXAMPLE 4a AND 4b

### (+)-6-[2(2,2-Dimethylpropyl)-2-hydroxy-4-(4-trifluoromethylphenyl)but-3-ynoylamino]-4-methyl-2,3-benzoxazin-1-one 4a and (−)-6-[2(2,2-dimethylpropyl)-2-hydroxy-4-(4-trifluoromethylphenyl)but-3-ynoylamino]-4-methyl-2,3-benzoxazin-1-one 4b

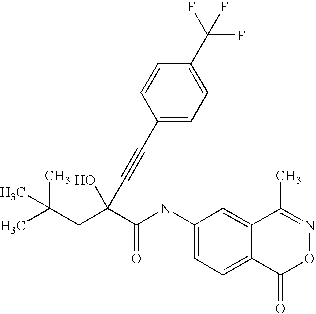

The racemic mixture obtained in Example 4 was separated by preparative chiral HPLC (column: Chiralpak AD 250×10 mm) into enantiomers 4a and 4b.

4a: [α]^{D}₂₀: +13.7° (CHCl₃, 11.8 mg/1 ml; λ=589 nm)

4b: [α]^{D}₂₀: −13.3° (CHCl₃, 10.1 mg/1 ml; λ=589 nm)

### EXAMPLE 5

### rac-6-[4,4-Dimethyl-2-hydroxy-2-phenylpentanoylamino]-4-methyl-2,3-benzoxazin-1-one

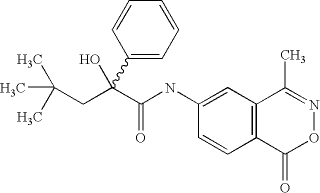

A 1 molar solution of phenylmagnesium bromide in tetrahydrofuran (1.32 ml) was diluted with 2 ml of absolute tetrahydrofuran. The mixture was cooled to −70° C. and then a solution of 200 mg of 6-(4,4-dimethyl-2-oxovaleroylamino)-4-methyl-2,3-benzoxazin-1-one in 5 ml of tetrahydrofuran was added. Subsequently, the mixture was left to stir at −70° C. for 3.5 hours. Thereafter, the reaction mixture was poured onto ice-cold saturated ammonium chloride solution. It was extracted with ethyl acetate. The combined organic phases were washed with saturated sodium chloride solution and dried over sodium sulphate. The crude product was chromatographed on silica gel. 169 mg of product were obtained.

¹H NMR (ppm, CDCl₃, 300 MHz): 1.07 (9H), 2.10 (1H), 2.59 (3H), 2.77 (1H), 2.97 (1H), 7.30-7.45 (3H), 7.68-7.79 (3H), 8.30 (1H), 8.34 (1H), 9.32 (1H).

### EXAMPLE 6

### rac-6-[4,4-Dimethyl-2-hydroxy-2-(phenylmethyl)pentanoylamino]-4-methyl-2,3-benzoxazin-1-one

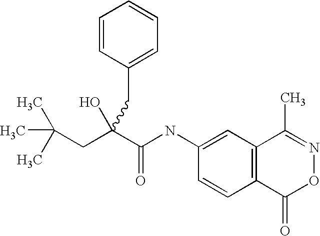

A 2 molar solution of benzylmagnesium chloride in tetrahydrofuran (665 μl) was diluted with 2 ml of absolute tetrahydrofuran. The mixture was cooled to −70° C. and then a solution of 200 mg of 6-(4,4-dimethyl-2-oxovaleroylamino)-4-methyl-2,3-benzoxazin-1-one in 5 ml of tetrahydrofuran was added. Subsequently, the mixture was left to stir at −70° C. for 1.5 hours. Thereafter, the reaction mixture was poured onto ice-cold saturated ammonium chloride solution. It was extracted with ethyl acetate. The combined organic phases were washed with saturated sodium chloride solution and dried over sodium sulphate. The crude product was chromatographed on silica gel. 116 mg of product were obtained.

¹H NMR (ppm, CDCl₃, 300 MHz): 1.00 (9H), 1.64 (1H), 2.26 (1H), 2.35 (1H), 2.59 (3H), 2.78 (1H), 2.31 (1H), 7.18 (2H), 7.22-7.32 (3H), 7.59 (1H), 8.19 (1H), 8.28 (1H), 8.87 (1H).

### EXAMPLE 6a AND 6b

(+)-6-[4,4-Dimethyl-2-hydroxy-2-(phenylmethyl)pentanoylamino]-4-methyl-2,3-benzoxazin-1-one 6a and (−)-6-[4,4-dimethyl-2-hydroxy-2-(phenylmethyl)pentanoylamino]-4-methyl-2,3-benzoxazin-1-one 6b

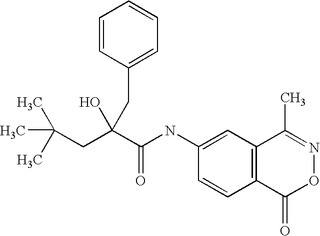

The racemic mixture obtained under Example 6 was separated by preparative chiral HPLC (column: Chiralpak AD 250×10 mm) into enantiomers 6a and 6b.

6a: [α]^{D}₂₀: +142.0° (CHCl₃, 10.1 mg/1 ml; λ=589 nm)

6b: [α]^{D}₂₀: −133.8° (CHCl₃, 10.2 mg/1 ml; λ=589 nm)

### Preparation of 6-(4-[1,3]dioxolan-2-yl-4-methyl-2-oxoyaleroylamino)-4-methyl-2,3-benzoxazin-1-one as starting material for the preparation of Examples 7 and 8

### a) 3-(tert-Butyldiphenylsilanyloxy)-2,2-dimethylpropan-1-ol

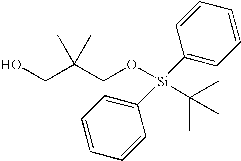

To a suspension of NaH (3.99 g) in absolute tetrahydrofuran was added a solution of 10.4 g of 2,2-dimethylpropane-1,3-diol in 100 ml of absolute tetrahydrofuran at 0° C. The mixture was left to stir at 23° C. for 45 minutes and then a solution of 26 ml of tert-butyldiphenylsilyl chloride in 30 ml of absolute tetrahydrofuran was added. The mixture was left to stir at 23° C. for 1.5 hours. Thereafter, the reaction mixture was poured onto saturated aqueous sodium hydrogencarbonate solution. The mixture was stirred for a further 10 minutes and then extracted with ethyl ether. The organic phase was washed with saturated aqueous sodium chloride solution, dried over sodium sulphate and concentrated under reduced pressure. The resulting crude product (35 g) was used directly in the next stage.

### b) 3-(tert-Butyldiphenylsilanyloxy)-2,2-dimethylpropionaldehyde

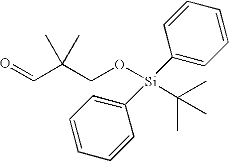

The compound (35 g) described under a) was dissolved in 500 ml of dichloromethane. With gentle cooling, 70 ml of triethylamine and 250 ml of dimethyl sulphoxide were then added and the mixture was stirred for a further 3 minutes. Subsequently, 40 g of sulphur trioxide-pyridine complex were added. The mixture was left to stir at 23° C. for 2 hours. The reaction mixture was then poured onto saturated aqueous ammonium chloride solution. The mixture was left to stir for a further 30 minutes and then extracted with dichloromethane. The organic phase was washed with saturated aqueous sodium chloride solution, dried over sodium sulphate and concentrated under reduced pressure. The resulting crude product (34 g) was used without purification in the next stage.

### c) tert-Butyl(2-[1,3]dioxolan-2-yl-2-methylpropoxy)diphenylsilane

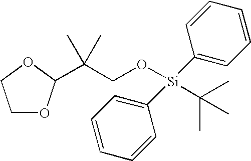

The crude product obtained under b) was dissolved in 300 ml of benzene. 80 ml of ethylene glycol and 2.5 g of p-toluenesulfonic acid were added, and the mixture was boiled on a water separator under reflux for 5 hours. Thereafter, the reaction mixture was poured onto saturated aqueous sodium hydrogencarbonate solution. The mixture was extracted with ethyl acetate, then the organic phase was washed with saturated aqueous sodium chloride solution, dried over sodium sulphate and concentrated under reduced pressure. The resulting crude product was chromatographed on silica gel. 28 g of product were obtained.

¹H NMR (ppm, CDCl₃, 300 MHz): 0.95 (6H), 1.05 (9H), 3.51 (2H), 3.80-3.93 (2H), 4.82 (1H), 7.32-7.48 (6H), 7.65-7.73 (4H).

### d) 2-[1,3]Dioxolan-2-yl-2-methylpropan-1-ol

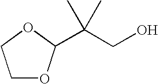

The product obtained under c) (28 g) was dissolved in tetrahydrofuran. Tetrabutylammonium fluoride was added and the mixture was left to stir at 40° C. for 2.5 hours. Thereafter, the reaction mixture was poured onto saturated aqueous sodium hydrogencarbonate solution. The mixture was stirred for a further 15 minutes and then extracted with ethyl acetate. The organic phase was washed with saturated aqueous sodium chloride solution, dried over sodium sulphate and concentrated under reduced pressure. The resulting crude product was chromatographed on silica gel. 8.88 g of product were obtained.

¹H NMR (ppm, CDCl₃, 400 MHz): 0.94 (6H), 2.56 (1H), 3.47 (2H), 3.83-4.00 (4H), 4.11 (1H).

### e) 2-[1,3]Dioxolan-2-yl-2-methylpropionaldehyde

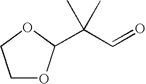

The product obtained under d) (8.88 g) was oxidized in analogy to the process described under b) with sulphur trioxide-pyridine complex, dimethyl sulphoxide, triethylamine in dichloromethane. The resulting crude product was chromatographed on silica gel. 6.2 g of product were obtained.

¹H NMR (ppm, CDCl₃, 400 MHz): 1.11 (6H), 3.85-4.00 (4H), 4.83 (1H), 9.65 (1H).

### f) 4-[1,3]Dioxolan-2-yl-4-methylpent-2-enoic acid ethyl ester

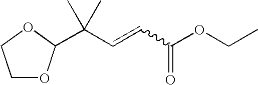

To a suspension of sodium hydride (2.58 g) in 30 ml of dimethoxyethane was added slowly, at 0° C., a solution of 12.91 ml of 2-(diethoxyphosphoryl)acetic acid ethyl ester in 40 ml of dimethoxyethane. The mixture was left to stir at 0° C. for a further 1 hour and then a solution of 6.2 g of the substance described under e) in 40 ml of dimethoxyethane was added. Thereafter, the mixture was allowed to come to 23° C. and stirred at this temperature for 2.5 hours. Subsequently, the reaction mixture was poured onto saturated aqueous ammonium chloride solution. The mixture was left to stir for a further 15 minutes and then extracted with ethyl acetate. The organic phase was washed with saturated aqueous sodium chloride solution, dried over sodium sulphate and concentrated under reduced pressure. The resulting crude product was chromatographed on silica gel. 8.67 g of product were obtained.

¹H NMR (ppm, CDCl₃, 400 MHz): 1.09 (6H), 1.28 (3H), 3.80-3.97 (4H), 4.18 (2H), 4.63 (1H), 5.84 (1H), 7.00 (1H).

### g) 4-[1,3]Dioxolan-2-yl-4-methylpentanoic acid ethyl ester

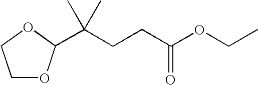

A solution of the substance described under f) (8.67 g) in 100 ml of a 1:1 mixture of ethanol and tetrahydrofuran was hydrogenated under standard pressure in the presence of palladium-carbon. After filtering off with suction and concentrating, 8.25 g of the crude product were obtained, which were used in the next stage without further purification.

### h) 4-[1,3]Dioxolan-2-yl-2-hydroxy-4-methylpentanoic acid ethyl ester

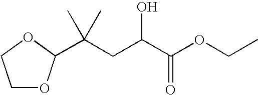

To a solution of 2 g of the compound prepared under g) in 20 ml of absolute tetrahydrofuran, 26.15 ml of a 0.5 molar solution of potassium hexamethyldisilazide in toluene were added at −70° C. The mixture was left to stir at −70° C. for a further 30 minutes and then a solution of 3.4 g of 2-phenylsulphonyl-3-phenyloxaziridine in 35 ml of absolute tetrahydrofuran was added slowly. Subsequently, the mixture was left to stir at −70° C. for a further hour and then poured onto saturated aqueous ammonium chloride solution. The mixture was left to stir for a further 30 minutes and then extracted with ethyl acetate. The organic phase was washed with saturated aqueous sodium chloride solution and dried over sodium sulphate. The resulting crude product was extracted by stirring with a mixture of diisopropyl ether and hexane. Subsequently, the mixture was filtered with suction, the crystals were discarded and the mother liquor was concentrated under reduced pressure. The resulting crude product was chromatographed on silica gel. 1.92 g of product were obtained.

### ¹H NMR (ppm, CDCl₃, 300 MHz): 1.00 (3H), 1.03 (3H), 1.29 (3H), 1.65-1.85 (2H), 3.44 (1H), 3.81-4.02 (4H), 4.20 (2H), 4.32 (1H), 4.61 (1H).

### i) 4-[1,3]Dioxolan-2-yl-4-methyl-2-oxopentanoic acid ethyl ester

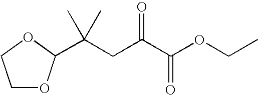

To a solution of 8 g of the compound described under h) in 100 ml of dichloromethane were added 100 ml of a 0.35 molar solution of 1,1-dihydro-1,1,1-triacetoxy-1,2-benzodioxol-3(1H)-one (Dess-Martin periodane) in dichloromethane. The mixture was left to stir at 23° C. for a further 14 hours. Subsequently, it was diluted with 500 ml of methyl tert-butyl ether and then poured onto 1 l of an aqueous solution of 34 g of sodium hydrogencarbonate and 100 g of sodium thiosulphate. The mixture was left to stir for 30 minutes, then the phases were separated and the aqueous phase was extracted with methyl tert-butyl ether. The combined organic phases were washed with saturated aqueous sodium hydrogencarbonate solution and saturated aqueous sodium chloride solution, and dried over sodium sulphate. The resulting crude product (7.7 g) was used without further purification in the next stage.

### j) 4-[1,3]Dioxolan-2-yl-4-methyl-2-oxopentanoic acid

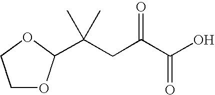

To a solution of the compounds described under i) (7.7 g) in 230 ml of ethanol was added a solution of 13.5 g of sodium hydroxide in 115 ml of water. The mixture was left to stir at 23° C. for 14 hours, then diluted with water and extracted with ethyl acetate. Subsequently, the aqueous phase was acidified with two normal hydrochloric acid (pH 4). Thereafter, it was extracted with ethyl acetate and the organic phase was washed with saturated aqueous sodium chloride solution. It was dried over sodium sulphate and concentrated under reduced pressure. The resulting crude product (4.05 g) was used in the next stage without purification.

### k) 6-(4-[1,3]Dioxolan-2-yl-4-methyl-2-oxovaleroylamino)-4-methyl-2,3-benzoxazin-1-one

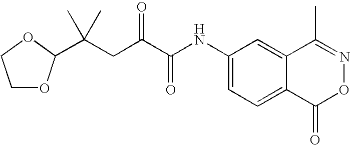

4.05 g of the carboxylic acid described under j) were dissolved in 100 ml of N,N-dimetylacetamide. At −10° C., 1.53 ml of thionyl chloride were added and the mixture was left to stir at −10° C. for a further hour. Subsequently, 4.59 g of 6-amino-4-methyl-2,3-benzoxazin-1-one were added in portions. Thereafter, the mixture was left to stir for 3 hours (−10° C. to 0° C.). Subsequently, the reaction mixture was poured onto ice-water. It was extracted with ethyl acetate. The organic phase was washed with saturated aqueous sodium chloride solution, dried over sodium sulphate and concentrated under reduced pressure. The crude product was purified by column chromatography on silica gel with a mixture of hexane/ethyl acetate. 2.52 g of product were obtained.

¹H NMR (ppm, CDCl₃, 300 MHz): 1.12 (6H), 2.59 (3H), 2.92 (2H), 3.75-3.90 (4H), 4.59 (1H), 7.88 (1H), 8.30 (1H), 8.38 (1H), 9.15 (1H).

### EXAMPLE 7

### rac-6-[4,4-Dimethyl-2,5-dihydroxy-2-phenylpentanoylamino]-4-methyl-2,3-benzoxazin-1-one

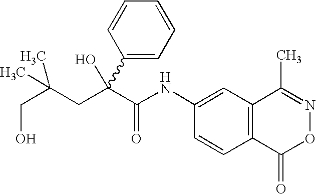

### a) rac-6-[4-[1,3]Dioxolan-2-yl-2-hydroxy-4-methyl-2-phenylvaleroylamino]-4-methyl-2,3-benzoxazin-1-one

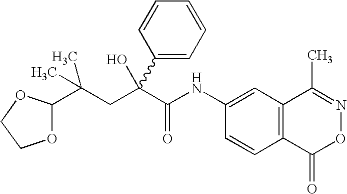

6-(4-[1,3]Dioxolan-2-yl-4-methyl-2-oxovaleroylamino)-4-methyl-2,3-benzoxazin-1-one (500 mg) was reacted with phenylmagnesium bromide in tetrahydrofuran in analogy to Example 5. After column chromatography, 317 mg of product were obtained.

¹H NMR (ppm, CDCl₃, 400 MHz): 0.95 (3H), 1.08 (3H), 2.25 (1H), 2.56 (3H), 2.71 (1H), 3.92-4.15 (4H), 4.50 (1H), 6.70 (1H), 7.28 (1H), 7.36 (2H), 7.68 (1H), 7.73 (2H), 8.28 (2H), 9.54 (1H).

### b) rac-6-[4,4-Dimethyl-2,5-dihydroxy-2-phenylpentanoylamino]-4-methyl-2,3-benzoxazin-1-one

The product obtained under 7a) (317 mg) was dissolved in 10 ml of acetone. 1 ml of 2 normal hydrochloric acid was added and the reaction mixture was left to boil under reflux for 14 h. Subsequently, the mixture was poured onto saturated aqueous sodium hydrogencarbonate solution and extracted with dichloromethane. The combined organic phases were washed with saturated aqueous sodium chloride solution, dried over sodium sulphate and concentrated under reduced pressure. The resulting crude product was dissolved in 5 ml of dimethoxyethane. The mixture was cooled to 0° C. 22 mg of sodium borohydride were then added. Subsequently, 250 μl of methanol were added slowly. The mixture was left to stir at 0° C. for 1.5 hours and then the reaction mixture was poured onto saturated aqueous ammonium chloride solution. The mixture was then extracted with ethyl acetate. The combined organic phases were washed with saturated aqueous sodium chloride solution, dried over sodium sulphate and concentrated under reduced pressure. The resulting crude product was purified by column chromatography on silica gel. 78 mg of product were obtained.

¹H NMR (ppm, CDCl₃, 400 MHz): 0.79 (3H), 1.04 (3H), 2.20 (1H), 2.53 (3H), 2.70 (1H), 3.50 (2H), 7.26 (1H), 7.35 (2H), 7.68 (1H), 7.77 (2H), 8.23 (1H), 8.30 (1H), 9.60 (1H).

### EXAMPLE 8

### rac-6-[4,4-Dimethyl-2,5-dihydroxy-2-(phenylmethyl)pentanoylamino]-4-methyl-2,3-benzoxazin-1-one

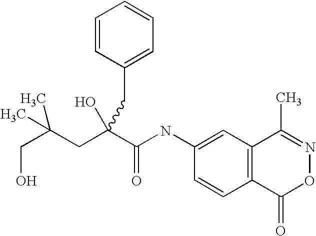

### a) rac-6-[2- Benzyl-4-[1,3]dioxolan-2-yl-2-hydroxy-4-methylvaleroylamino]-4-methyl-2,3-benzoxazin-1-one

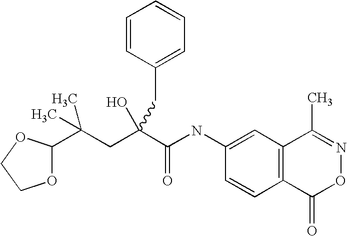

6-(4-[1,3]Dioxolan-2-yl-4-methyl-2-oxovaleroylamino)-4-methyl-2,3-benzoxazin-1-one (500 mg) was reacted with benzylmagnesium chloride in tetrahydrofuran in analogy to Example 6. After column chromatography, 390 mg of product were obtained.

¹H NMR (ppm, CDCl₃, 300 MHz): 0.99 (3H), 1.04 (3H), 2.08 (1H), 2.38 (1H), 2.56 (3H), 2.82 (1H), 3.04 (1H), 3.75-3.95 (4H), 4.46 (1H), 6.14 (1H), 7.12-7.25 (5H), 7.50 (1H), 8.09 (1H), 8.24 (1H), 9.10 (1H).

### b) rac-6-[4,4-Dimethyl-2,5-dihydroxy-2-(phenylmethyl)pentanoylamino]-4-methyl-2,3-benzoxazin-1-one

The compound described under 8a) was converted analogously to the process described under 7). After column chromatography on silica gel, 58 mg of product were obtained.

¹H NMR (ppm, CDCl₃, 400 MHz): 0.99 (6H), 1.81 (1H), 2.50-2.58 (4H), 2.86 (1H), 3.11 (1H), 3.40-3.50 (2H), 7.10-7.25 (5H), 7.48 (1H), 8.02 (1H), 8.20 (1H), 9.01 (1H).

### Preparation of 6-(3-cyclohexyl-2-oxopropanoylamino)-4-methyl-2,3-benzoxazin-1-one as starting material for the preparation of Examples 9, 10, 11 and 12

### l) 2-Hydroxy-3-cyclohexylpropanoic acid ethyl ester

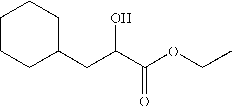

To a solution of ethyl 3-cyclohexylpropionate (0.4 g) in tetrahydrofuran (7 ml) was added, at −70° C., a solution of potassium hexamethyldisilizane (5.6 ml, 0.5 M in toluene). The mixture was left to stir at −70° C. for a further 30 minutes and then a solution of 3-phenyl-2-phenylsulphonyloxaziridine (0.5 g) in tetrahydrofuran (8 ml) was added. The mixture was left to stir at −70° C. for one hour. Thereafter, the reaction mixture was poured onto a saturated aqueous ammonium chloride solution. The mixture was stirred for a further 30 minutes and the phases were separated. The organic phase was washed with saturated aqueous sodium chloride solution, dried over sodium sulphate and concentrated under reduced pressure. The resulting crude product was chromatographed on silica gel. 0.3 g of product was obtained.

¹H NMR (ppm, CDCl₃, 400 MHz): 0.89-1.01 (2H), 1.10-1.25 (2H), 1.30 (3H), 1.48-1.73 (8H), 1.84 (1H), 4.20-4.26 (3H).

### m) 2-Oxo-3-cyclohexylpropanoic acid ethyl ester

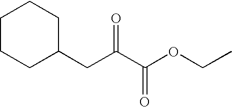

The product obtained under l) (0.3 g) was oxidized in analogy to the process described under i). The resulting crude product was chromatographed on silica gel. 0.22 g of product was obtained.

¹H NMR (ppm, CDCl₃, 400 MHz): 0.98 (2H), 1.10-1.29 (2H), 1.36 (3H), 1.58-1.71 (6H), 1.89 (1H), 2.70 (2H), 4.31 (2H).

### n) 2-Oxo-3-cyclohexylpropanoic acid

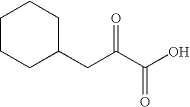

The product obtained under m) (1 g) was prepared in analogy to the process described under j). The resulting crude product (0.8 g) was used in the next stage without purification.

### o) 6-(3-Cyclohexyl-2-oxopropanoylamino)-4-methyl-2,3-benzoxazin-1-one

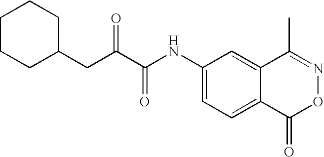

The crude product obtained under n) was prepared in analogy to the process described under k). The resulting crude product was chromatographed on silica gel. 1.3 g of product were obtained.

¹H NMR (ppm, CDCl₃, 400 MHz): 1.04-1.34 (4H), 1.58-1.77 (6H), 1.92-2.02 (1H), 2.61 (3H), 2.91 (2H), 4.31 (2H), 7.86 (1H), 8.34 (1H), 8.39 (1H), 9.17 (1H).

### EXAMPLE 9

### rac-6-[3-Cyclohexyl-2-hydroxy-2-(phenyl methyl)propanoylamino]-4-methyl-2,3-benzoxazin-1-one

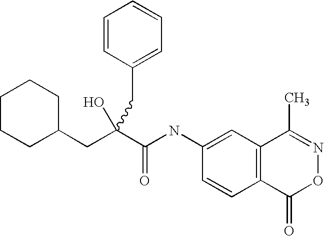

6-(3-Cyclohexyl-2-oxopropanoylamino)-4-methyl-2,3-benzoxazin-1-one (150 mg) was reacted with benzylmagnesium chloride in tetrahydrofuran in analogy to Example 6. After column chromatography, 62 mg of product were obtained.

¹H NMR (ppm, CDCl₃, 400 MHz): 0.97-1.26 (4H), 1.46-1.69 (7H), 1.77 (1H), 2.10 (1H), 2.24 (1H), 2.59 (3H), 2.91 (1H), 3.35 (1H), 7.18-7.31 (5H), 7.60 (1H), 8.23 (1H), 8.29 (1H), 8.85 (1H).

### EXAMPLE 9a AND 9b

### (+)-6-[3-Cyclohexyl-2-hydroxy-2-(phenylmethyl)propanoylamino]-4-methyl-2,3-benzoxazin-1-one 9a and (−)-6-[3-Cyclohexyl-2-hydroxy-2-(phenylmethyl)propanoylamino]-4-methyl-2,3-benzoxazin-1-one 9b

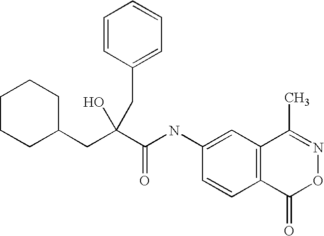

The racemic mixture obtained under Example 9 was separated by preparative chiral HPLC (column: Chiralpak AD 250×10 mm) into enantiomers 9a and 9b.

9a: [α]^{D}₂₀: +118.0° (CHCl₃, 9.5 mg/1 ml; λ=589 nm)

9b: [α]^{D}₂₀: −112.8° (CHCl₃, 9.2 mg/1 ml; λ=589 nm)

### EXAMPLE 10

### rac-6-[3-Cyclohexyl-2-hydroxy-2-phenylpropanoylamino]-4-methyl-2,3-benzoxazin-1-one

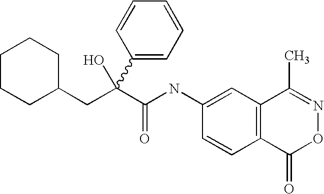

6-(3-Cyclohexyl-2-oxopropanoylamino)-4-methyl-2,3-benzoxazin-1-one (150 mg) was reacted with phenylmagnesium bromide in tetrahydrofuran in analogy to Example 5. After column chromatography, 76 mg of product were obtained.

¹H NMR (ppm, CDCl₃, 400 MHz): 1.01-1.22 (4H), 1.50-1.76 (7H), 2.11 (1H), 2.42 (1H), 2.55 (3H), 2.80 (1H), 7.29-7.42 (3H), 7.65-7.70 (3H), 8.29 (1H), 8.32 (1H), 9.19 (1H).

### EXAMPLE 11

### rac-6-[3-Cyclohexyl-2-hydroxy-2-(phenylethynyl)propanoylamino]-4-methyl-2,3-benzoxazin-1-one

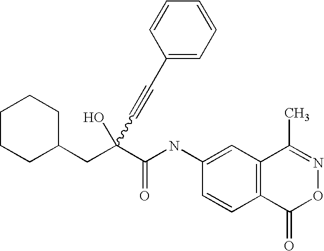

6-(3-cyclohexyl-2-oxopropanoylamino)-4-methyl-2,3-benzoxazin-1-one (150 mg) was reacted with phenylethyne and n-butyllithium in tetrahydrofuran in analogy to Example 2. After column chromatography, 120 mg of product were obtained.

¹H NMR (ppm, CDCl₃, 400 MHz): 1.06-1.33 (5H), 1.67-1.99 (6H), 2.04 (1H), 2.13 (1H), 2.59 (3H), 3.23 (1H), 7.29-7.37 (3H), 7.45 (2H), 7.73 (1H), 8.35 (1H), 8.40 (1H), 9.09 (1H).

### EXAMPLE 11a AND 11b

### (+)-6-[3-Cyclohexyl-2-hydroxy-2-(phenylethynyl)propanoylamino]-4-methyl-2,3-benzoxazin-1-one 11a and (−)-6-[3-Cyclohexyl-2-hydroxy-2-(phenylethynyl)propanoylamino]-4-methyl-2,3-benzoxazin-1-one 11b

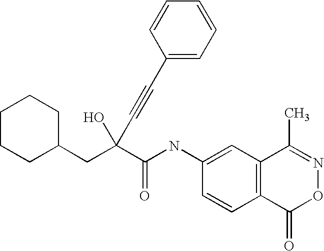

The racemic mixture obtained under Example 11 was separated by preparative chiral HPLC (column: Chiralpak AD 250×10 mm) into enantiomers 11a and 11b.

11a: [α]^{D}₂₀: +20.1° (CHCl₃, 9.8 mg/1 ml; λ=589 nm)

11b: [α]^{D}₂₀: −21.4° (CHCl₃, 10.2 mg/1 ml; λ=589 nm)

### EXAMPLE 12

### rac-6-[3-Cyclohexyl-2-hydroxy-2-((4-methylphenyl)ethynyl)propanoylamino]-4-methyl-2,3-benzoxazin-1-one

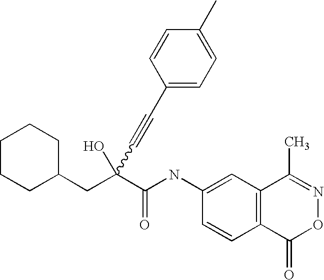

6-(3-Cyclohexyl-2-oxopropanoylamino)-4-methyl-2,3-benzoxazin-1-one (150 mg) was reacted with 4-methylphenylethyne and n-butyllithium in tetrahydrofuran in analogy to Example 2. After column chromatography, 170 mg of product were obtained.

¹H NMR (ppm, CDCl₃, 400 MHz): 1.05-1.33 (5H), 1.67-1.98 (7H), 2.12 (1H), 2.35 (3H), 2.59 (3H), 3.24 (1H), 7.13 (2H), 7.34 (2H), 7.73 (1H), 8.35 (1H), 8.39 (1H), 9.09 (1H).

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding German application No. 10 2006 061 913.7, filed Dec. 21, 2006, U.S. Provisional Application Ser. No. 60/880,706 filed Jan. 17, 2007, U.S. Provisional Application Ser. No. 60/944,870 filed Jun. 19, 2007, and U.S. Provisional Application Ser. No. 60/938,338 filed May 16, 2007, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Compounds of the general formula I 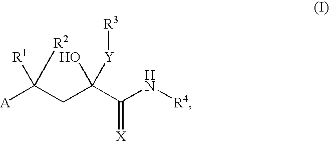
in which
A is hydrogen, a C₁-C₈-alkyl, C₂-C₈-alkenyl, C₂-C₈-alkynyl radical which is optionally mono- or polysubstituted identically or differently by Z, or a C₃-C₁₀-cycloalkyl or 3-12-membered heterocycloalkyl radical which is optionally mono- or polysubstituted identically or differently by M, or else is Z itself, where Z is defined as follows:
cyano, halogen, hydroxyl, nitro, —C(O)R^{b}, CO₂R^{b}, —O—R^{b}, —S—R^{b}, SO₂NR^{c}R^{d}, —C(O)—NR^{c}R^{d}, —OC(O)—NR^{c}R^{d}, C═NOR^{b}, —NR^{c}R^{d}, —PO₃(R^{b})₂, —NR^{e}COR^{b}, —NR^{e}CSR^{b}, —NR^{e}S(O)R^{b}, —NR^{e}S(O)₂R^{b}, —NR^{e}CONR^{c}R^{d}, —NR^{e}COOR^{b}, —NR^{e}C(NH)NR^{c}R^{d}, —NR^{e}CSNR^{c}R^{d}, —NR^{e}S(O)NR^{c}R^{d}, —NR^{e}S(O)₂NR^{c}R^{d}, —S(O)R , —S(O)NR^{c}R^{d}, —S(O)₂R^{b}, —SO₂OR^{b}, —CSNR^{c}R^{d}, —CR^{b}(OH)—R^{b} or
a C₃-C₁₀-cycloalkyl or 3-12-membered heterocycloalkyl radical which is optionally mono- or polysubstituted identically or differently by M and
M is C₁-C₆-alkyl or a —COR^{b}, CO₂R^{b}, —O—R^{b} or —NR^{c}R^{d} group, where
R^{b} is hydrogen or a C₁-C₆-alkyl, C₂-C₈-alkenyl, C₂-C₈-alkynyl, C₃-C₁₀-cycloalkyl, C₆-C₁₂-aryl or a partly or fully fluorinated C₁-C₃-alkyl radical and
R^{c} and R^{d} are each independently hydrogen, a C₁-C₆-alkyl, C₂-C₈-alkenyl, C₂-C₈-alkynyl, C₃-C₁₀-cycloalkyl, C₆-C₁₂-aryl radical; a C(O)R^{b} group where R^{b} is as defined above or a hydroxyl group,
where, when
R^{c} is a hydroxyl group, R^{d} can only be hydrogen, C₁-C₆-alkyl, C₂-C₈-alkenyl, C₂-C₈-alkynyl, C₃-C₁₀-cycloalkyl or C₆-C₁₂-aryl, and vice versa, and also
R^{e} is hydrogen, C₁-C₆-alkyl, C₂-C₈-alkenyl, C₂-C₈-alkynyl, C₃-C₁₀-cycloalkyl or C₆-C₁₂-aryl, and
R¹ and R² are each independently an unbranched or branched C₁-C₅-alkyl group or, together with the carbon atom of the chain, form a ring having a total of 3-7 members,
where, when
A is hydrogen, R¹ and R² cannot both be a methyl radical,
R³ is hydrogen or a C₁-C₈-alkyl, C₂-C₈-alkenyl, C₂-C₈-alkynyl, C₃-C₁₀-cycloalkyl, 3-12-membered heterocycloalkyl radical which is optionally mono- or polysubstituted identically or differently by K, or a mono- or bicyclic C₆-C₁₂-aryl or 3-12-membered heteroaryl radical which is optionally mono- or polysubstituted identically or differently by L, and
K is cyano, halogen, hydroxyl, nitro, —C(O)R^{b}, CO₂R^{b}, —O—R^{b}, —S—R^{b}, SO₂NR^{c}R^{d}, —C(O)—NR^{c}R^{d}, —OC(O)—N R^{c}R^{d}, —C═NOR^{b}, —NR^{c}R^{d} or a C₃-C₁₀-cycloalkyl, 3-12-membered heterocycloalkyl radical which is optionally mono- or polysubstituted identically or differently by M, or a C₆-C₁₂-aryl or 3-12-membered heteroaryl radical which is optionally mono- or polysubstituted by L, with the definition of M specified under A, and
L is C₁-C₈-alkyl, C₂-C₈-alkenyl, C₂-C₈-alkynyl, a partly or fully fluorinated C₁-C₆-alkyl, a partly or fully fluorinated C₁-C₆-alkoxy, C₁-C₆-alkoxy-C₁-C₆-alkyl, C₁-C₆-alkoxy-C₁-C₆-alkoxy, a mono- or bicyclic (CH₂)ₚ—C₃-C₁₀-cycloalkyl, a mono- or bicyclic 3-12-membered (CH₂)ₚ-heterocycloalkyl radical, (CH₂)ₚCN, (CH₂)ₚHal, (CH₂)ₚNO₂, a mono- or bicyclic (CH₂)ₚ—C₆-C₁₂-aryl radical, a mono- or bicyclic 3-12-membered (CH₂)ₚ-heteroaryl radical, or
—(CH₂)ₚPO₃(R^{b})₂, —(CH₂)ₚNR^{c}R^{d}, —(CH₂)ₚNR^{e}COR^{b}, —(CH₂)ₚNR^{e}CSR^{b}, —(CH₂)ₚNR^{e}S(O)R^{b}, —(CH₂)ₚNR^{e}S(O)₂R^{b}, —(CH₂)ₚNR^{e}CONR^{c}R^{d}, —(CH₂)ₚNR^{e}COOR^{b}, —(CH₂)ₚNR^{e}C(NH)NR^{c}R^{d}, —(CH₂)ₚNR^{e}CSNR^{c}R^{d}, —(CH₂)ₚNR^{e}S(O)NR^{c}R^{d}, —(CH₂)ₚNR^{e}S(O)₂NR^{c}R^{d}, —(CH₂)ₚCOR , —(CH₂)ₚCSR , —(CH₂)ₚ S(O)R^{b}, —(CH₂)ₚS(O)(NH)R^{b}, —(CH₂)ₚS(O)₂R^{b}, —(CH₂)ₚS(O)₂NR^{c}R^{d}, —(CH₂)ₚSO₂OR^{b} , —(CH₂)ₚCO₂R^{b}, —(CH₂)ₚCONR^{c}R^{d}, —(CH₂)ₚCSNR^{c}R^{d}, —(CH₂)ₚOR^{b}, —(CH₂)ₚSR^{b}, —(CH₂)ₚCR^{b}(OH)—R^{b}, —(CH₂)ₚ—C═NOR^{b}, —O—(CH₂)ₙ—O—, —O—(CH₂)ₙ—CH₂—, —O—CH═CH— or —(CH₂)ₙ₊₂₋
and the terminal oxygen atoms and/or carbon atoms are linked to directly adjacent ring carbon atoms and
n is 1 or 2 and
p is 0, 1, 2, 3, 4, 5 or 6, and
X is one oxygen atom and two hydrogen atoms
Y is (CH₂)ₘ, —C≡C— or —CH═CH— where
m=0 or 1, and
R⁴ is an aromatic or heteroaromatic 3- to 12-membered mono- or bicycle which is unsubstituted or optionally substituted by from 1 to 3 of the radicals mentioned under L, or one of the following groups mentioned under B or C:
B: 6-membered/6-membered ring systems: 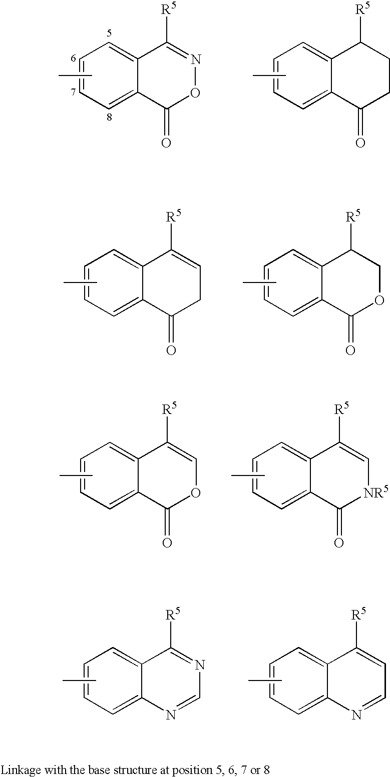
C: 6-membered/5-membered ring systems: 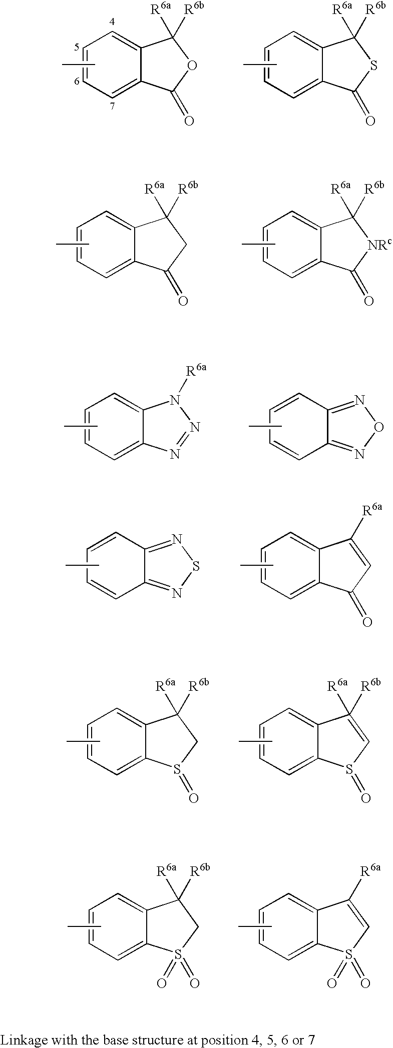
where
R⁵ is hydrogen or C₁-C₄-alkyl, or a partly or fully fluorinated C₁-C₄-alkyl,
R^{6a} and R^{6b} are each independently hydrogen, C₁-C₄-alkyl or a partly or fully fluorinated C₁-C₄-alkyl, or, together with the ring carbon atom, form a 3- to 6-membered ring,
and the pharmaceutically acceptable salts thereof.

2. Compounds according to claim 1, in which A is a C₁-C₈-alkyl radical substituted identically or differently by Z or a C₃-C₁₀-cycloalkyl radical optionally mono- or polysubstituted identically or differently by M, and Z and M are each as defined under claim 1.

3. Compounds according to claim 2, in which A is C₁-C₈-alkyl or C₃-C₁₀-cycloalkyl, while R¹ and R² are each methyl.

4. Compounds according to claim 1, in which A is hydrogen, and R¹ and R² together with the carbon atom of the chain, form a ring composed of 3-7 carbon atoms.

5. Compounds according to claim 4, in which R¹ and R² together are cyclopentyl or cyclohexyl rings.

6. Compounds according to claim 1, in which R³ is C₁-C₈-alkyl, a mono- or bicyclic C₆-C₁₂-aryl radical or a 3-12-membered heteroaryl radical, with the prerequisite that Y is —C≡C— or —CH═CH—.

7. Compounds according to claim 1, in which R³ is a mono- or bicyclic C₆-C₁₂-aryl radical or a 3-12-membered heteroaryl radical, with the prerequisite that Y is (CH₂)ₘ.

8. Compounds according to claim 1, in which R⁴is a mono- or disubstituted mono- or bicyclic aromatic or one of the B groups specified for R⁴ under claim 1 with linkage at position 6, or one of the C groups with linkage at position 5.

9. Compounds according to claim 8, in which R⁴ has one of the following definitions:

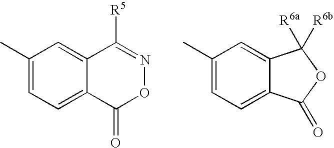

10. Compounds according to claim 1, in which R⁵ is methyl or ethyl.

11. Compounds according to claim 1, in which R⁶ is hydrogen.

12. Compounds according to claim 1, in which p is 0, 1 or 2.

13. Compounds according to claim 1, in which L is C₁-C₈-alkyl, C₂-C₈-alkenyl, C₂-C₈-alkynyl, partly or fully fluorinated C₁-C₆-alkyl, —(CH₂)ₚCN, (CH₂)ₚHal, (CH₂)ₚNO₂, (CH₂)ₚ—C₆-C₁₂-aryl, —(CH₂)ₚ-heteroaryl, —(CH₂)ₚNR^{c}R^{d}, —(CH₂)ₚNR^{e}COR^{b}, —(CH₂)ₚNR^{e}S(O)₂R^{b}, —(CH₂)ₚNR^{e}CONR^{c}R^{d}, —(CH₂)ₚNR^{e}S(O)NR^{c}R^{d}, —(CH₂)ₚNR^{e}S(O)₂NR^{c}R^{d}, —(CH₂)ₚCOR^{b}, —(CH₂)ₚS(O)R^{b}, —(CH₂)ₚS(O)₂R^{b}, —(CH₂)ₚS(O)₂NR^{c}R^{d}, —(CH₂)ₚCO₂R^{b}, —(CH₂)ₚCONR^{c}R^{d}, —(CH₂)ₚOR^{b}, —(CH₂)ₚCR^{b}(OH)—R^{b} with the definition of R^{b}, R^{c}, R^{d} and R^{e} specified under claim 1.

14. Compounds according to claim 1, in which Z is cyano, halogen, hydroxyl, nitro,
—C(O)R^{b}, CO₂R^{b}, —O—R^{b}, —SO₂NR^{c}R^{d}, —C(O)—NR^{c}R^{d}, —NR^{c}R^{d}, —NR^{e}COR^{b}, NR^{e}S(O)R^{b}, —NR^{e}S(O)₂R^{b}, —NR^{e}CONR^{c}R^{d}, —S(O)R^{b}, —S(O)NR^{c}R^{d}, —S(O)₂R^{b}, —CR^{b}(OH)—R^{b} or a C₃-C₁₀-cycloalkyl or heterocycloalkyl optionally mono- or polysubstituted identically or differently by M, with the definition of R^{b}, R^{c}, R^{d}, R^{e} and M specified under claim 1.

15. Compounds according to claim 1, specifically

| | | |
|---|---|---|
| | Racemic or | |
| No. | enantiomer | —Y—R¹ |

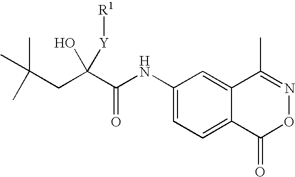
| | | |
|---|---|---|
| 123 | rac+− | 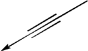 |
| 456 | rac+− | 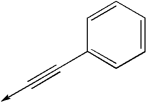 |
| 789 | rac+− | 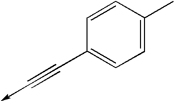 |
| 101112 | rac+− | 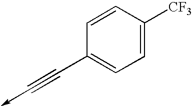 |
| 131415 | rac+− | 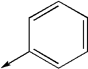 |
| 161718 | rac+− | 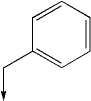 |
| 192021 | rac+− | 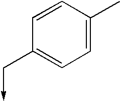 |
| 222324 | rac+− | 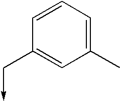 |

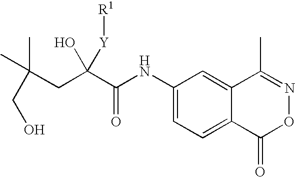
| | | |
|---|---|---|
| 252627 | rac+− | 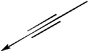 |
| 282930 | rac+− | 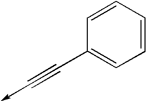 |
| 313233 | rac+− | 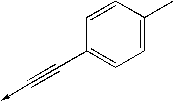 |
| 343536 | rac+− | 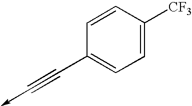 |
| 373839 | rac+− | 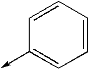 |
| 404142 | rac+− | 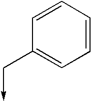 |
| 434445 | rac+− | 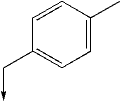 |
| 464748 | rac+− | 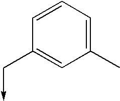 |

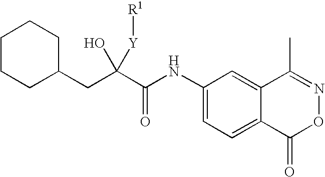
| | | |
|---|---|---|
| 495051 | rac+− | 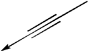 |
| 525354 | rac+− | 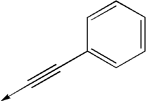 |
| 555657 | rac+− | 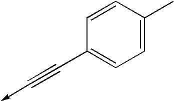 |
| 585960 | rac+− | 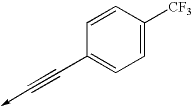 |
| 616263 | rac+− | 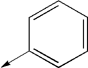 |
| 646566 | rac+− | 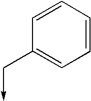 |
| 676869 | rac+− | 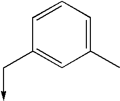 |
| 707172 | rac+− | 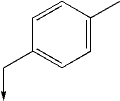 |

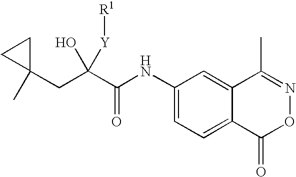
| | | |
|---|---|---|
| 737475 | rac+− | 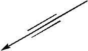 |
| 767778 | rac+− | 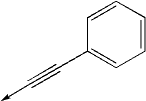 |
| 798081 | rac+− | 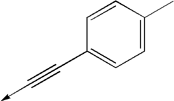 |
| 828384 | rac+− | 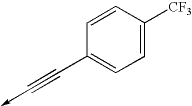 |
| 858687 | rac+− | 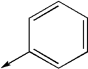 |
| 888990 | rac+− | 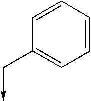 |
| 919293 | rac+− | 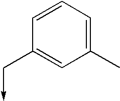 |
| 949596 | rac+− | 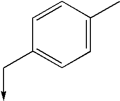 |

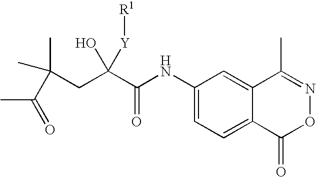
| | | |
|---|---|---|
| 979899 | rac+− | 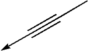 |
| 100101102 | rac+− | 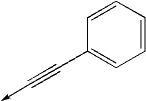 |
| 103104105 | rac+− | 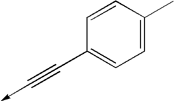 |
| 106107108 | rac+− | 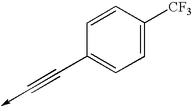 |
| 109110111 | rac+− | 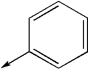 |
| 112113114 | rac+− | 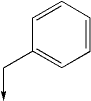 |
| 115116117 | rac+− | 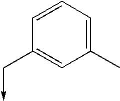 |
| 118119120 | rac+− | 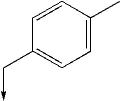 |

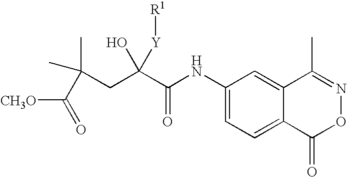
| | | |
|---|---|---|
| 121122123 | rac+− | 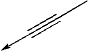 |
| 124125126 | rac+− | 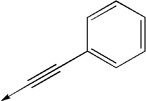 |
| 127128129 | rac+− | 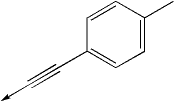 |
| 130131132 | rac+− | 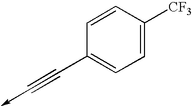 |
| 133134135 | rac+− | 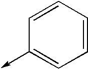 |
| 136137138 | rac+− | 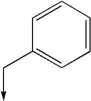 |
| 139140141 | rac+− | 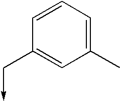 |
| 142143144 | rac+− | 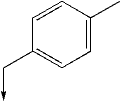 |

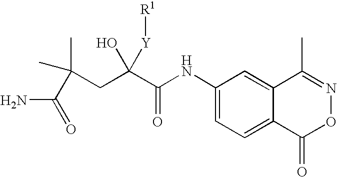
| | | |
|---|---|---|
| 145146147 | rac+− | 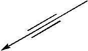 |
| 148149150 | rac+− | 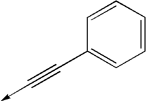 |
| 151152153 | rac+− | 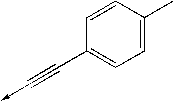 |
| 154155156 | rac+− | 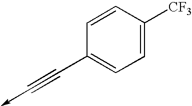 |
| 157158159 | rac+− | 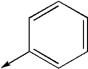 |
| 160161162 | rac+− | 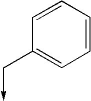 |
| 163164165 | rac+− | 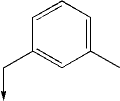 |
| 166167168 | rac+− | 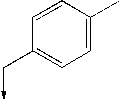 |

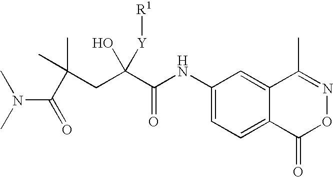
| | | |
|---|---|---|
| 169170171 | rac+− | 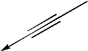 |
| 172173174 | rac+− | 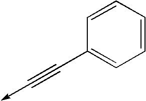 |
| 175176177 | rac+− | 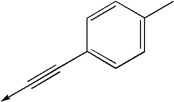 |
| 178179180 | rac+− | 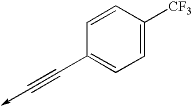 |
| 181182183 | rac+− | 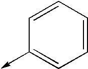 |
| 184185186 | rac+− | 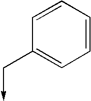 |
| 187188189 | rac+− | 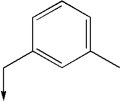 |
| 190191192 | rac+− | 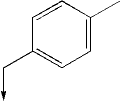 |

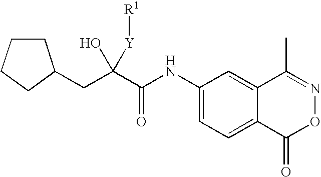
| | | |
|---|---|---|
| 193194195 | rac+− | 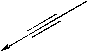 |
| 196197198 | rac+− | 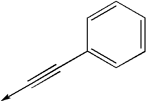 |
| 199200201 | rac+− | 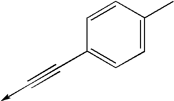 |
| 202203204 | rac+− | 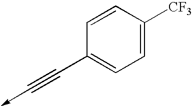 |
| 205206207 | rac+− | 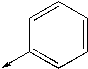 |
| 208209210 | rac+− | 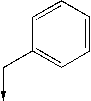 |
| 211212213 | rac+− | 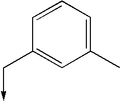 |
| 214215216 | rac+− | 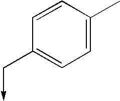 |

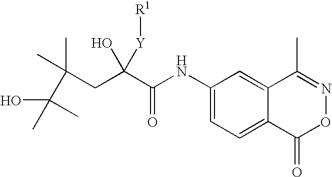
| | | |
|---|---|---|
| 217218219 | rac+− | 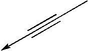 |
| 220221222 | rac+− | 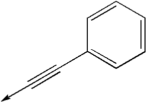 |
| 223224225 | rac+− | 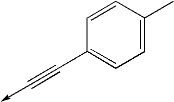 |
| 226227228 | rac+− | 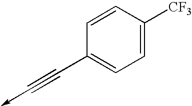 |
| 229230231 | rac+− | 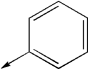 |
| 232233234 | rac+− | 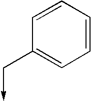 |
| 235236237 | rac+− | 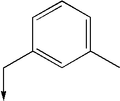 |
| 238239240 | rac+− | 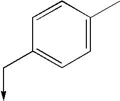 |

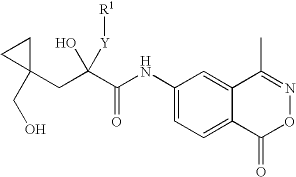
| | | |
|---|---|---|
| 241242243 | rac+− | 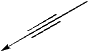 |
| 244245246 | rac+− | 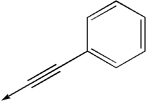 |
| 247248249 | rac+− | 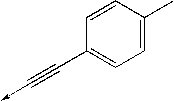 |
| 250251252 | rac+− | 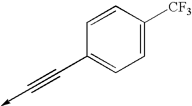 |
| 253254255 | rac+− | 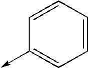 |
| 256257258 | rac+− | 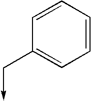 |
| 259260261 | rac+− | 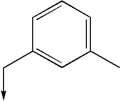 |
| 262263264 | rac+− | 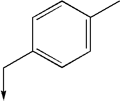 |

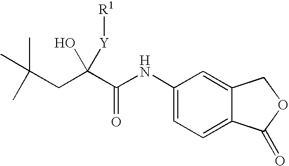
| | | |
|---|---|---|
| 265266267 | rac+− | 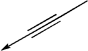 |
| 268269270 | rac+− | 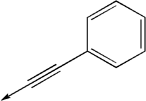 |
| 271272273 | rac+− | 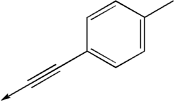 |
| 274275276 | rac+− | 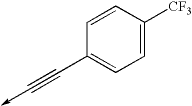 |
| 277278279 | rac+− | 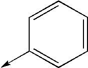 |
| 280281282 | rac+− | 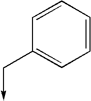 |
| 283284285 | rac+− | 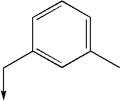 |
| 286287288 | rac+− | 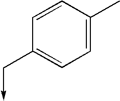 |

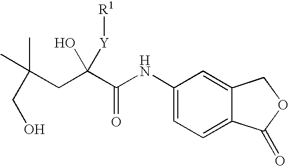
| | | |
|---|---|---|
| 289290291 | rac+− | 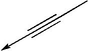 |
| 292293294 | rac+− | 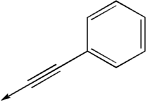 |
| 295296297 | rac+− | 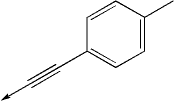 |
| 298299300 | rac+− | 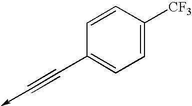 |
| 301302303 | rac+− | 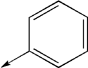 |
| 304305306 | rac+− | 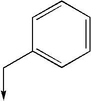 |
| 307308309 | rac+− | 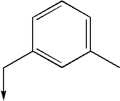 |
| 310311312 | rac+− | 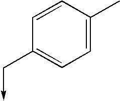 |

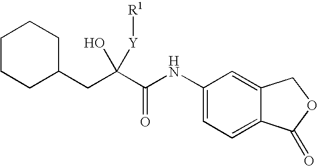
| | | |
|---|---|---|
| 313314315 | rac+− | 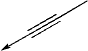 |
| 316317318 | rac+− | 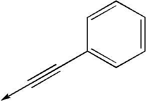 |
| 319320321 | rac+− | 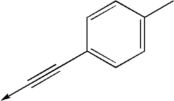 |
| 322323324 | rac+− | 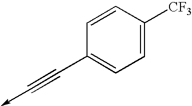 |
| 325326327 | rac+− | 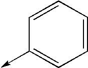 |
| 328329330 | rac+− | 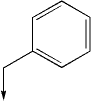 |
| 331332333 | rac+− | 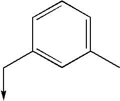 |
| 334335336 | rac+− | 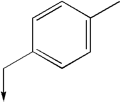 |

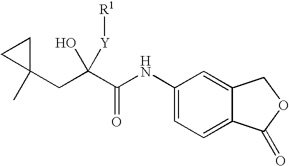
| | | |
|---|---|---|
| 337338339 | rac+− | 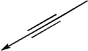 |
| 340341342 | rac+− | 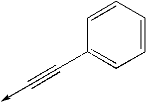 |
| 343344345 | rac+− | 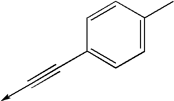 |
| 346347348 | rac+− | 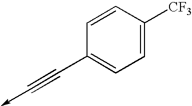 |
| 349350351 | rac+− | 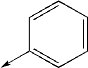 |
| 352353354 | rac+− | 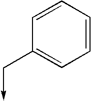 |
| 355356357 | rac+− | 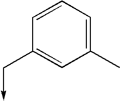 |
| 358359360 | rac+− | 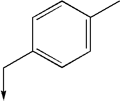 |

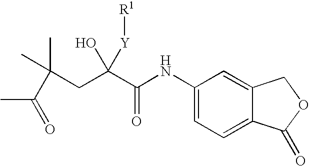
| | | |
|---|---|---|
| 361362363 | rac+− | 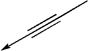 |
| 364365366 | rac+− | 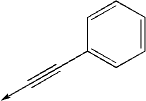 |
| 367368369 | rac+− | 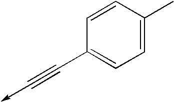 |
| 370371372 | rac+− | 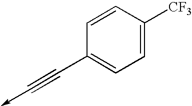 |
| 373374375 | rac+− | 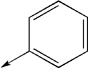 |
| 376377378 | rac+− | 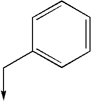 |
| 379380381 | rac+− | 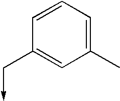 |
| 382383384 | rac+− | 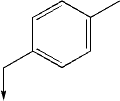 |

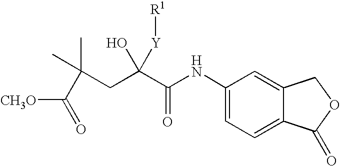
| | | |
|---|---|---|
| 385386387 | rac+− | 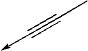 |
| 388389390 | rac+− | 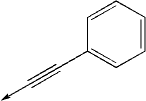 |
| 391392393 | rac+− | 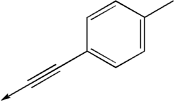 |
| 394395396 | rac+− | 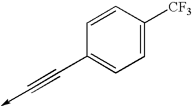 |
| 397398399 | rac+− | 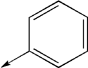 |
| 400401402 | rac+− | 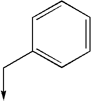 |
| 403404405 | rac+− | 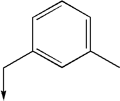 |
| 406407408 | rac+− | 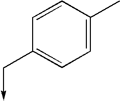 |

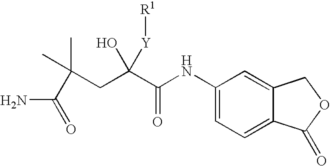
| | | |
|---|---|---|
| 409410411 | rac+− | 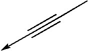 |
| 412413414 | rac+− | 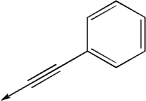 |
| 415416417 | rac+− | 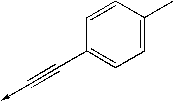 |
| 418419420 | rac+− | 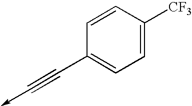 |
| 421422423 | rac+− | 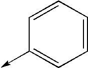 |
| 424425426 | rac+− | 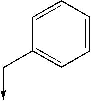 |
| 427428429 | rac+− | 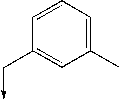 |
| 430431432 | rac+− | 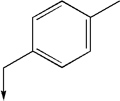 |

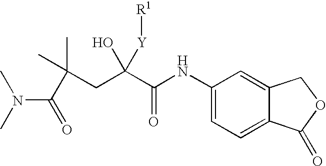
| | | |
|---|---|---|
| 433434435 | rac+− | 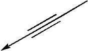 |
| 436437438 | rac+− | 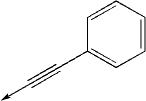 |
| 439440441 | rac+− | 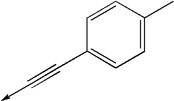 |
| 442443444 | rac+− | 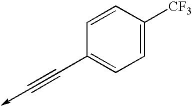 |
| 445446447 | rac+− | 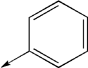 |
| 448449450 | rac+− | 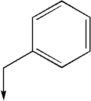 |
| 451452453 | rac+− | 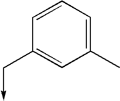 |
| 454455456 | rac+− | 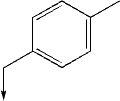 |

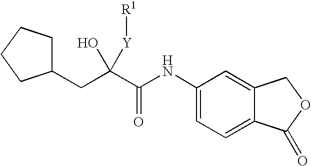
| | | |
|---|---|---|
| 457458459 | rac+− | 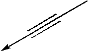 |
| 460461462 | rac+− | 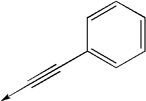 |
| 463464465 | rac+− | 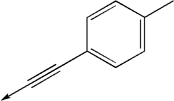 |
| 466467468 | rac+− | 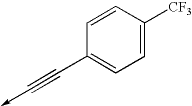 |
| 469470471 | rac+− | 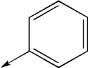 |
| 472473474 | rac+− | 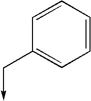 |
| 475476477 | rac+− | 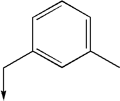 |
| 478479480 | rac+− | 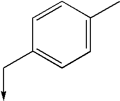 |

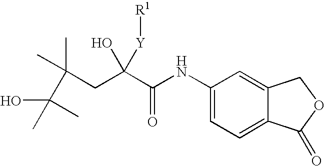
| | | |
|---|---|---|
| 481482483 | rac+− | 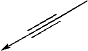 |
| 484485486 | rac+− | 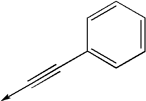 |
| 487488489 | rac+− | 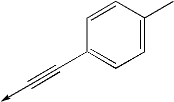 |
| 490491492 | rac+− | 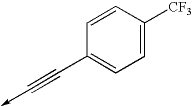 |
| 493494495 | rac+− | 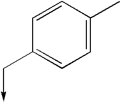 |
| 496497498 | rac+− | 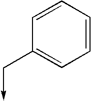 |
| 499599501 | rac+− | 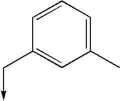 |
| 502503504 | rac+− | 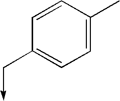 |

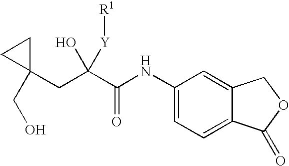
| | | |
|---|---|---|
| 505506507 | rac+− | 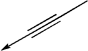 |
| 508509510 | rac+− | 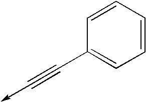 |
| 511512513 | rac+− | 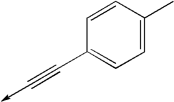 |
| 514515516 | rac+− | 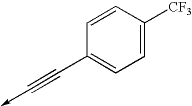 |
| 517518519 | rac+− | 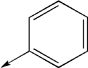 |
| 520521522 | rac+− | 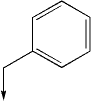 |
| 523524525 | rac+− | 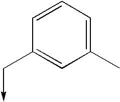 |
| 526527528 | rac+− | 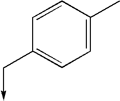 |

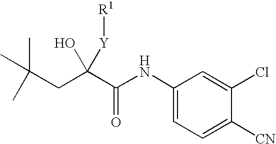
| | | |
|---|---|---|
| 529530531 | rac+− | 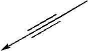 |
| 532533534 | rac+− | 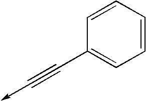 |
| 535536537 | rac+− | 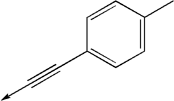 |
| 538539540 | rac+− | 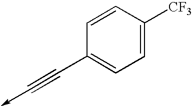 |
| 541542543 | rac+− | 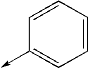 |
| 544545546 | rac+− | 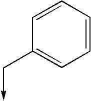 |
| 547548549 | rac+− | 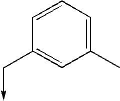 |
| 550551552 | rac+− | 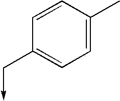 |

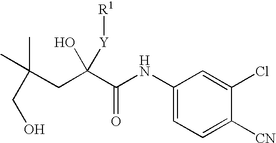
| | | |
|---|---|---|
| 553554555 | rac+− | 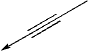 |
| 556557558 | rac+− | 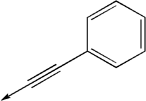 |
| 559560561 | rac+− | 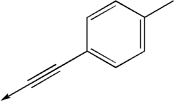 |
| 562563564 | rac+− | 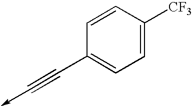 |
| 565566567 | rac+− | 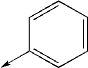 |
| 568569570 | rac+− | 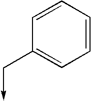 |
| 571572573 | rac+− | 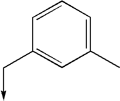 |
| 574575576 | rac+− | 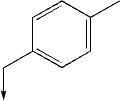 |

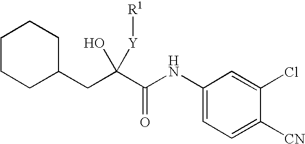
| | | |
|---|---|---|
| 577578579 | rac+− | 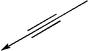 |
| 580581582 | rac+− | 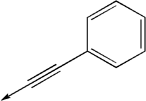 |
| 583584585 | rac+− | 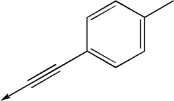 |
| 586587588 | rac+− | 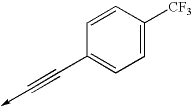 |
| 589590591 | rac+− | 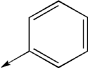 |
| 592593594 | rac+− | 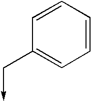 |
| 595596597 | rac+− | 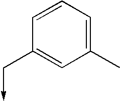 |
| 598599600 | rac+− | 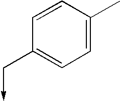 |

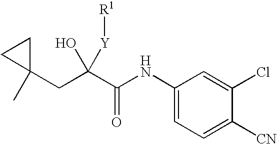
| | | |
|---|---|---|
| 601602603 | rac+− | 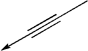 |
| 604605606 | rac+− | 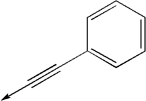 |
| 607608609 | rac+− | 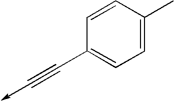 |
| 610611612 | rac+− | 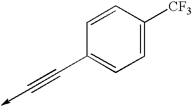 |
| 613614615 | rac+− | 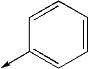 |
| 616617618 | rac+− | 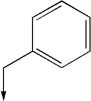 |
| 619620621 | rac+− | 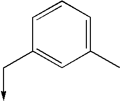 |
| 622623624 | rac+− | 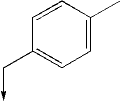 |

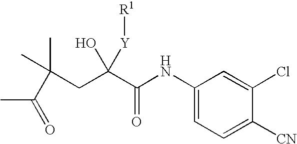
| | | |
|---|---|---|
| 625626627 | rac+− | 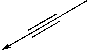 |
| 628629630 | rac+− | 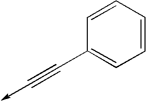 |
| 631632633 | rac+− | 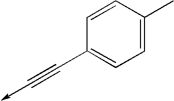 |
| 634635636 | rac+− | 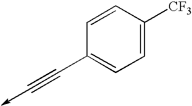 |
| 637638639 | rac+− | 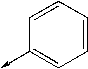 |
| 640641642 | rac+− | 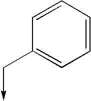 |
| 643644645 | rac+− | 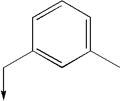 |
| 646647648 | rac+− | 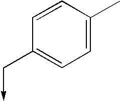 |

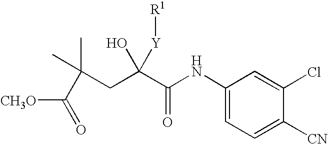
| | | |
|---|---|---|
| 649650651 | rac+− | 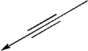 |
| 652653654 | rac+− | 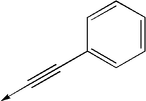 |
| 655656657 | rac+− | 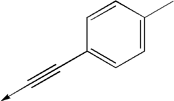 |
| 658659660 | rac+− | 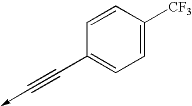 |
| 661662663 | rac+− | 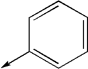 |
| 664665666 | rac+− | 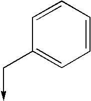 |
| 667668669 | rac+− | 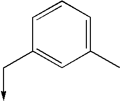 |
| 670671672 | rac+− | 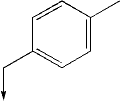 |

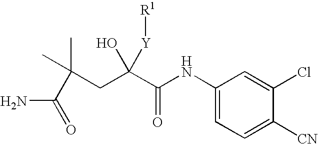
| | | |
|---|---|---|
| 673674675 | rac+− | 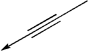 |
| 676677678 | rac+− | 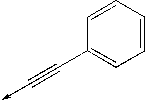 |
| 679680681 | rac+− | 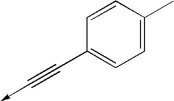 |
| 682683684 | rac+− | 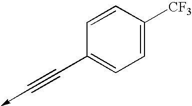 |
| 685686687 | rac+− | 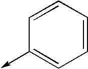 |
| 688689690 | rac+− | 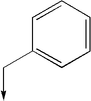 |
| 691692693 | rac+− | 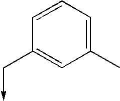 |
| 694695696 | rac+− | 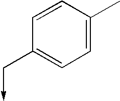 |

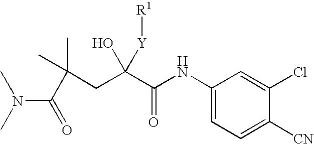
| | | |
|---|---|---|
| 697698699 | rac+− | 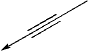 |
| 700701702 | rac+− | 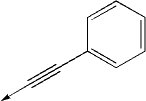 |
| 703704705 | rac+− | 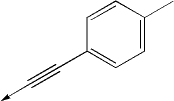 |
| 706707708 | rac+− | 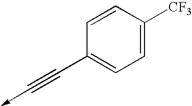 |
| 709710711 | rac+− | 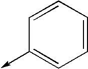 |
| 712713714 | rac+− | 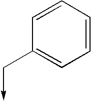 |
| 715716717 | rac+− | 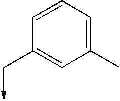 |
| 718719720 | rac+− | 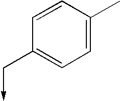 |

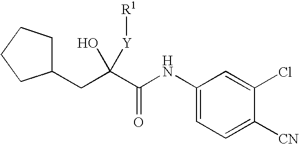
| | | |
|---|---|---|
| 721722723 | rac+− | 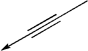 |
| 724725726 | rac+− | 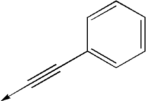 |
| 727728729 | rac+− | 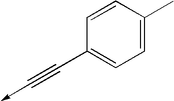 |
| 730731732 | rac+− | 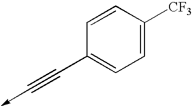 |
| 733734735 | rac+− | 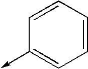 |
| 736737738 | rac+− | 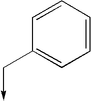 |
| 739740741 | rac+− | 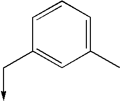 |
| 742743744 | rac+− | 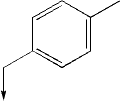 |

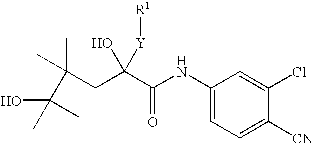
| | | |
|---|---|---|
| 745746747 | rac+− | 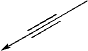 |
| 748749750 | rac+− | 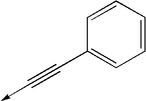 |
| 751752753 | rac+− | 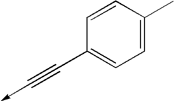 |
| 754755756 | rac+− | 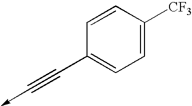 |
| 757758759 | rac+− | 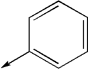 |
| 760761762 | rac+− | 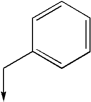 |
| 763764765 | rac+− | 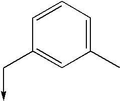 |
| 766767768 | rac+− | 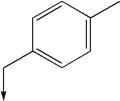 |

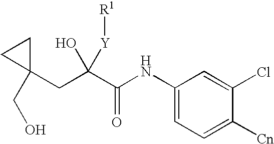
| | | |
|---|---|---|
| 769770771 | rac+− | 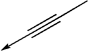 |
| 772773774 | rac+− | 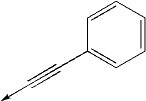 |
| 775776777 | rac+− | 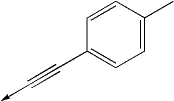 |
| 778779780 | rac+− | 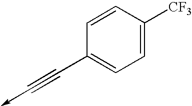 |
| 781782783 | rac+− | 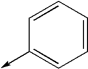 |
| 784785786 | rac+− | 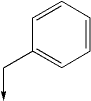 |
| 787788789 | rac+− | 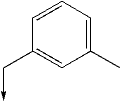 |
| 790791792 | rac+− | 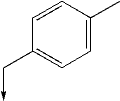 |

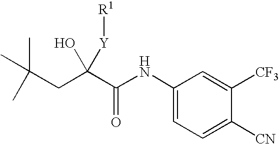
| | | |
|---|---|---|
| 793794795 | rac+− | 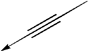 |
| 796797798 | rac+− | 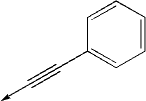 |
| 799800801 | rac+− | 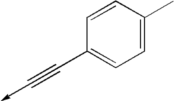 |
| 802803804 | rac+− | 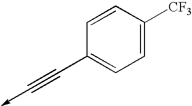 |
| 805806807 | rac+− | 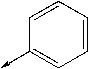 |
| 808809810 | rac+− | 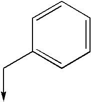 |
| 811812813 | rac+− | 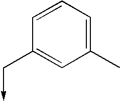 |
| 814815816 | rac+− | 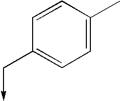 |

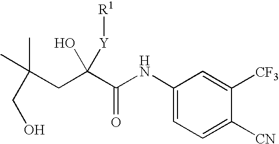
| | | |
|---|---|---|
| 817818819 | rac+− | 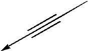 |
| 820821822 | rac+− | 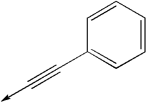 |
| 823824825 | rac+− | 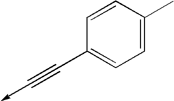 |
| 826827828 | rac+− | 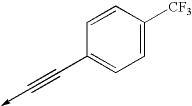 |
| 829830831 | rac+− | 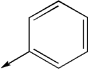 |
| 832833834 | rac+− | 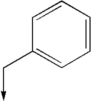 |
| 835836837 | rac+− | 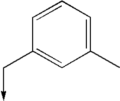 |
| 838839840 | rac+− | 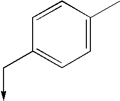 |

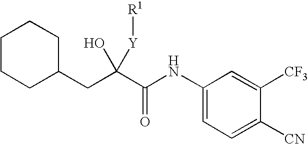
| | | |
|---|---|---|
| 841842843 | rac+− | 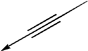 |
| 844845846 | rac+− | 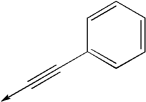 |
| 847848849 | rac+− | 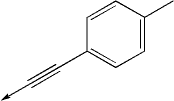 |
| 850851852 | rac+− | 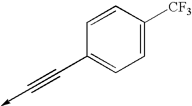 |
| 853854855 | rac+− | 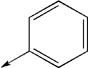 |
| 856857858 | rac+− | 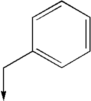 |
| 859860861 | rac+− | 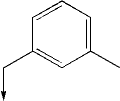 |
| 862863864 | rac+− | 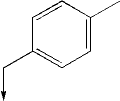 |

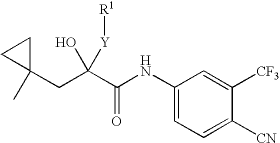
| | | |
|---|---|---|
| 865866867 | rac+− | 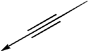 |
| 868869870 | rac+− | 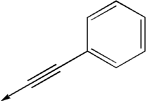 |
| 871872873 | rac+− | 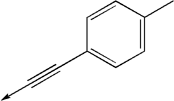 |
| 874875876 | rac+− | 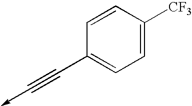 |
| 877878879 | rac+− | 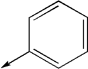 |
| 880881882 | rac+− | 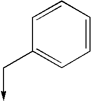 |
| 883884885 | rac+− | 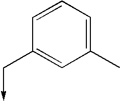 |
| 886887888 | rac+− | 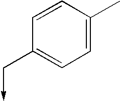 |

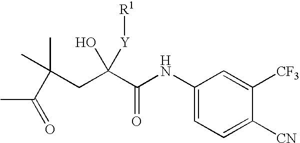
| | | |
|---|---|---|
| No. | enantiomer | —Y-R¹ |
| 889890891 | rac+− | 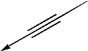 |
| 892893894 | rac+− | 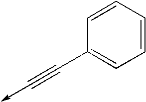 |
| 895896897 | rac+− | 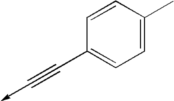 |
| 898899900 | rac+− | 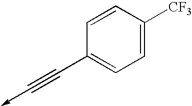 |
| 901902903 | rac+− | 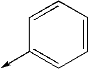 |
| 904905906 | rac+− | 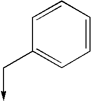 |
| 907908909 | rac+− | 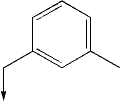 |
| 910911912 | rac+− | 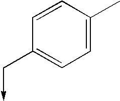 |

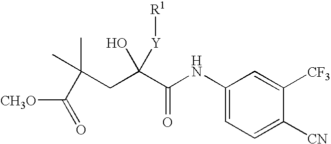
| | | |
|---|---|---|
| 913914915 | rac+− | 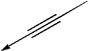 |
| 916917918 | rac+− | 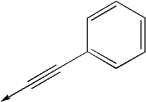 |
| 919920921 | rac+− | 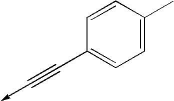 |
| 922923924 | rac+− | 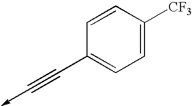 |
| 925926927 | rac+− | 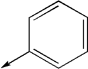 |
| 928929930 | rac+− | 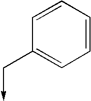 |
| 931932933 | rac+− | 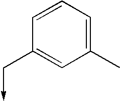 |
| 934935936 | rac+− | 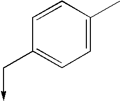 |

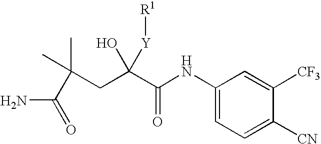
| | | |
|---|---|---|
| 937938939 | rac+− | 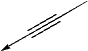 |
| 940941942 | rac+− | 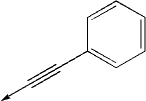 |
| 943944945 | rac+− | 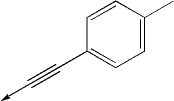 |
| 946947948 | rac+− | 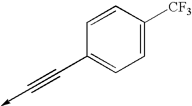 |
| 949950951 | rac+− | 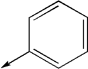 |
| 952953954 | rac+− | 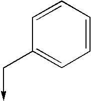 |
| 955956957 | rac+− | 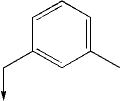 |
| 958959960 | rac+− | 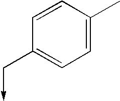 |

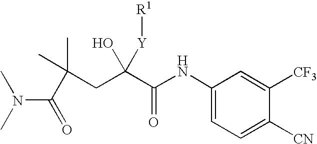
| | | |
|---|---|---|
| 961962963 | rac+− | 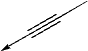 |
| 964965966 | rac+− | 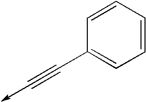 |
| 967968969 | rac+− | 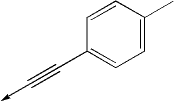 |
| 970971972 | rac+− | 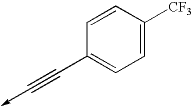 |
| 973974975 | rac+− | 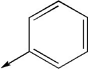 |
| 976977978 | rac+− | 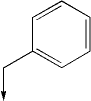 |
| 979980981 | rac+− | 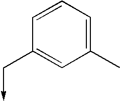 |
| 982983984 | rac+− | 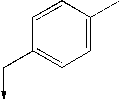 |

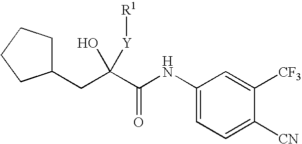
| | | |
|---|---|---|
| 985986987 | rac+− | 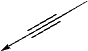 |
| 988989990 | rac+− | 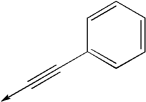 |
| 991992993 | rac+− | 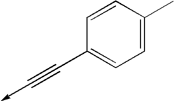 |
| 994995996 | rac+− | 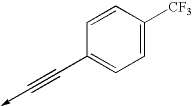 |
| 997998999 | rac+− | 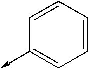 |
| 100010011002 | rac+− | 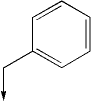 |
| 100310041005 | rac+− | 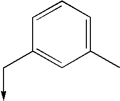 |
| 100610071008 | rac+− | 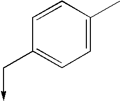 |

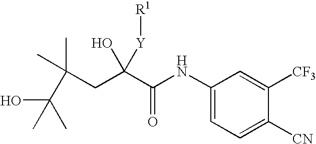
| | | |
|---|---|---|
| 100910101011 | rac+− | 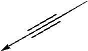 |
| 101210131014 | rac+− | 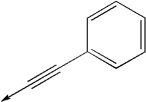 |
| 101510161017 | rac+− | 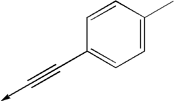 |
| 101810191020 | rac+− | 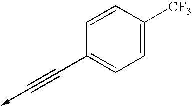 |
| 102110221023 | rac+− | 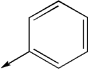 |
| 102410251026 | rac+− | 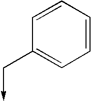 |
| 102710281029 | rac+− | 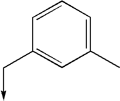 |
| 103010311032 | rac+− | 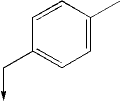 |

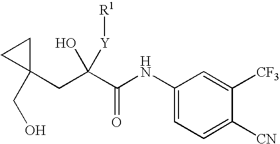
| | | |
|---|---|---|
| 103310341035 | rac+− | 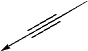 |
| 103610371038 | rac+− | 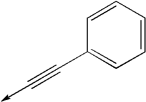 |
| 103910401041 | rac+− | 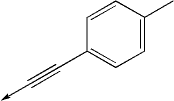 |
| 104210431044 | rac+− | 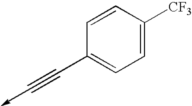 |
| 104510461047 | rac+− | 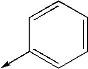 |
| 104810491050 | rac+− | 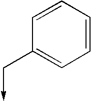 |
| 105110521053 | rac+− | 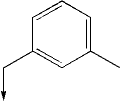 |
| 105410551056 | rac+− | 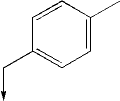 |

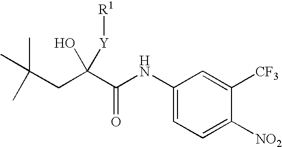
| | | |
|---|---|---|
| 105710581059 | rac+− | 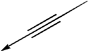 |
| 106010611062 | rac+− | 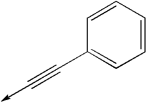 |
| 106310641065 | rac+− | 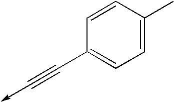 |
| 106610671068 | rac+− | 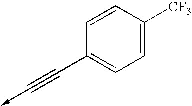 |
| 106910701071 | rac+− | 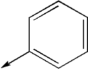 |
| 107210731074 | rac+− | 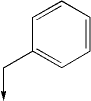 |
| 107510761077 | rac+− | 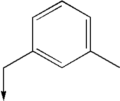 |
| 107810791080 | rac+− | 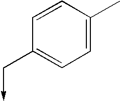 |

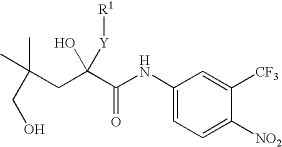
| | | |
|---|---|---|
| 108110821083 | rac+− | 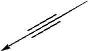 |
| 108410851086 | rac+− | 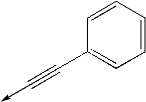 |
| 108710881089 | rac+− | 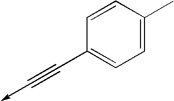 |
| 109010911092 | rac+− | 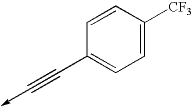 |
| 109310941095 | rac+− | 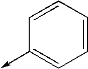 |
| 109610971098 | rac+− | 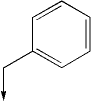 |
| 109911001101 | rac+− | 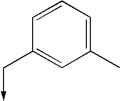 |
| 110211031104 | rac+− | 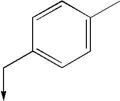 |

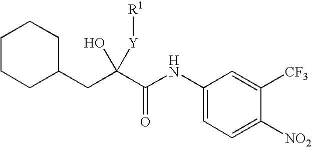
| | | |
|---|---|---|
| 110511061107 | rac+− | 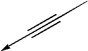 |
| 110811091110 | rac+− | 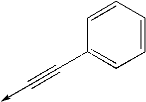 |
| 111111121113 | rac+− | 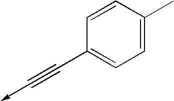 |
| 111411151116 | rac+− | 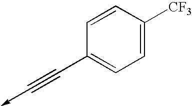 |
| 111711181119 | rac+− | 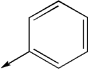 |
| 112011211122 | rac+− | 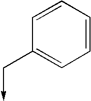 |
| 112311241125 | rac+− | 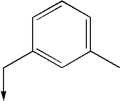 |
| 112611271128 | rac+− | 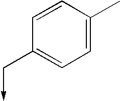 |

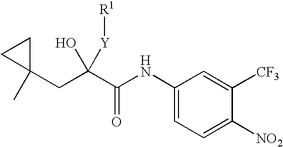
| | | |
|---|---|---|
| 112911301131 | rac+− | 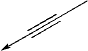 |
| 113211331134 | rac+− | 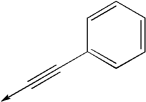 |
| 113511361137 | rac+− | 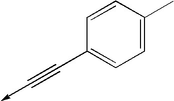 |
| 113811391140 | rac+− | 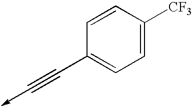 |
| 114111421143 | rac+− | 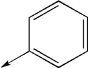 |
| 114411451146 | rac+− | 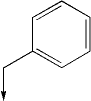 |
| 114711481149 | rac++ | 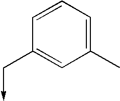 |
| 115011511152 | rac+− | 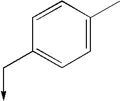 |

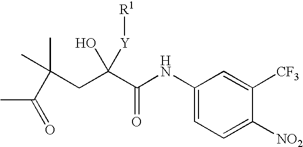
| | | |
|---|---|---|
| 115311541155 | rac+− | 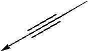 |
| 115611571158 | rac+− | 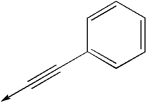 |
| 115911601161 | rac+− | 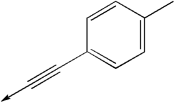 |
| 116211631164 | rac+− | 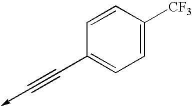 |
| 116511661167 | rac+− | 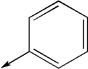 |
| 116811691170 | rac+− | 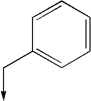 |
| 117111721173 | rac+− | 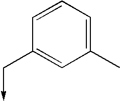 |
| 117411751176 | rac+− | 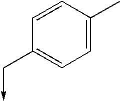 |

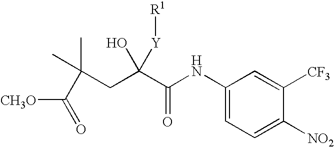
| | | |
|---|---|---|
| 117711781179 | rac+− | 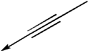 |
| 118011811182 | rac+− | 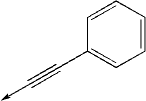 |
| 118311841185 | rac+− | 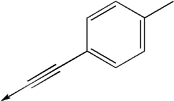 |
| 118611871188 | rac+− | 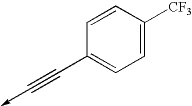 |
| 118911901191 | rac+− | 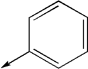 |
| 119211931194 | rac+− | 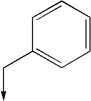 |
| 119511961197 | rac+− | 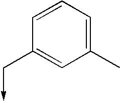 |
| 119811991200 | rac+− | 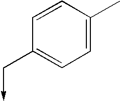 |

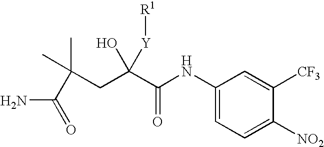
| | | |
|---|---|---|
| 120112021203 | rac+− | 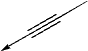 |
| 120412051206 | rac+− | 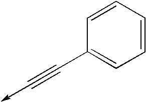 |
| 120712081209 | rac+− | 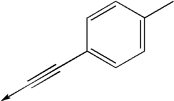 |
| 121012111212 | rac+− | 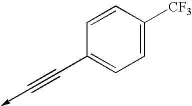 |
| 121312141215 | rac+− | 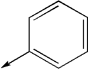 |
| 121612171218 | rac+− | 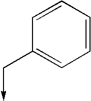 |
| 121912201221 | rac+− | 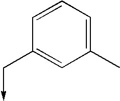 |
| 122212231224 | rac+− | 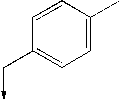 |

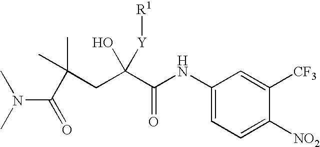
| | | |
|---|---|---|
| 122512261227 | rac+− | 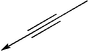 |
| 122812291230 | rac+− | 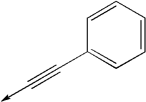 |
| 123112321233 | rac+− | 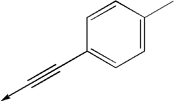 |
| 123412351236 | rac+− | 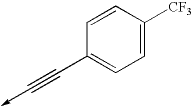 |
| 123712381239 | rac+− | 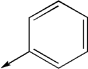 |
| 124012411242 | rac+− | 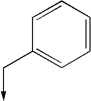 |
| 124312441245 | rac+− | 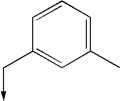 |
| 124612471248 | rac+− | 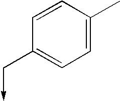 |

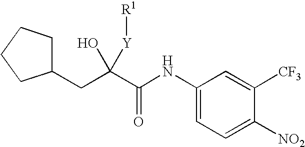
| | | |
|---|---|---|
| 124912501251 | rac+− | 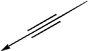 |
| 125212531254 | rac+− | 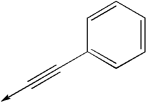 |
| 125512561257 | rac+− | 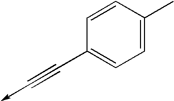 |
| 125812591260 | rac+− | 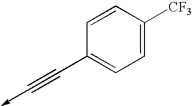 |
| 126112621263 | rac+− | 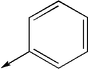 |
| 126412651266 | rac+− | 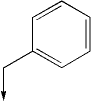 |
| 126712681269 | rac+− | 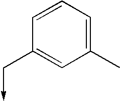 |
| 127012711272 | rac+− | 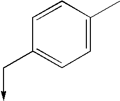 |

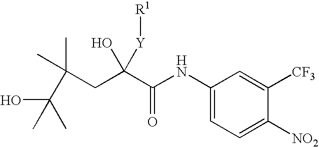
| | | |
|---|---|---|
| 127312741275 | rac+− | 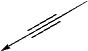 |
| 127612771278 | rac+− | 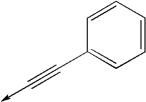 |
| 127912801281 | rac+− | 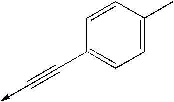 |
| 128212831284 | rac+− | 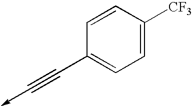 |
| 128512861287 | rac+− | 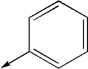 |
| 128812891290 | rac+− | 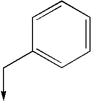 |
| 129112921293 | rac+− | 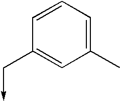 |
| 129412951296 | rac+− | 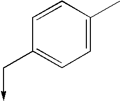 |

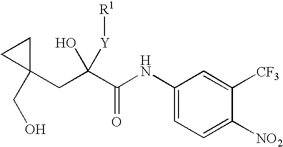
| | | |
|---|---|---|
| 129712981299 | rac+− | 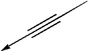 |
| 130013011302 | rac+− | 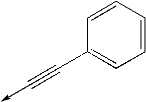 |
| 130313041305 | rac+− | 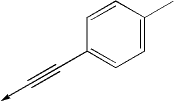 |
| 130613071308 | rac+− | 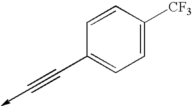 |
| 130913101311 | rac+− | 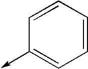 |
| 131213131314 | rac+− | 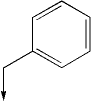 |
| 131513161317 | rac+− | 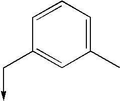 |
| 131813191320 | rac+− | 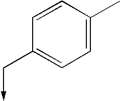 |

16. Pharmaceutical composition comprising at least one compound of the general formula I according to claim 1 and, where appropriate, at least one further active ingredient together with pharmaceutically suitable excipients and/or carriers.

17. Pharmaceutical composition according to claim 16, where the further active ingredient is a SERM (selective estrogen receptor modulator), an aromatase inhibitor, antiestrogen or a prostaglandin.

18. Pharmaceutical composition according to claim 17, where the further active ingredients may be tamoxifen, 5-(4-{5-[(RS)-(4,4,5,5,5-pentafluropentyl)sulfinyl]pentyloxy}phenyl)-6-phenyl-8,9-dihydro-7H-benzocyclohepten-2-ol, ICI 182 780 (7alpha-[9-(4,4,5,5-pentafluropentylsulphinyl)nony]estra-1,3,5(10)-triene-3,17beta-diol), 11beta-fluoro-7alpha-[5-(methyl{3-[(4,4,5,5,5-pentafluoropentyl)sulphanyl]-propyl}amino)pentyl]estra-1,3,5(10)-triene-3,17beta-diol, 11beta-fluoro-7alpha-{5-[methyl(7,7,8,8,9,9,10,10,10-nonafluorodecyl)amino]pentyl}estra-1,3,5(10)-triene-3,17beta-diol, 11beta-fluoro-17alpha-methyl-7alpha-5-[methyl(8,8,9,9,9-pentafluorononyl)amino]pentyl}estra-1,3,5(10)-triene-3,17beta-diol, clomifene, raloxifene, fadrozole, formestane, letrozole, anastrozole or atamestane.

19. Compounds according to claim 1 for the manufacture of a medicament.

20. A method for the therapy and/or prophylaxis of gynaecological disorders such as. endometriosis, leiomyomas of the uterus, dysfunctional bleeding and dysmenorrhoea, comprising administering a compound according to claim 1.

21. A method according to claim 1 for the therapy and/or prophylaxis of hormone-dependent tumours.

22. A method according claim 1 for the therapy and/or prophylaxis of breast carcinomas.

23. A method according to claim 1 for the therapy and/or prophylaxis of endometrial carcinoma.

24. A method according to claim 1 for the therapy and/or prophylaxis of ovarian carcinomas.

25. A method according to claim 1 for the therapy and/or prophylaxis of prostate carcinomas.

26. A method according to claim 1 for female hormone replacement therapy.

27. A method according claim 1 for female fertility control.
